(19) United States
(12) Patent Application Publication
(10) Pub. No.: US 2006/0239970 A1
(43) Pub. Date: Oct. 26, 2006

(54) Herpesvirus amplicon particles

(76) Inventors: Howard J. Federoff, Rochester, NY (US); Marc W. Halterman, Penfield, NY (US); William J. Bowers, Webster, NY (US)

Correspondence Address: FISH & RICHARDSON PC, P.O. BOX 1022, MINNEAPOLIS, MN, 55440-1022, US

(21) Appl. No.: 10/543,216

(22) PCT Filed: Jan. 23, 2004
(86) PCT No.: PCT/US04/01821

§ 371 Date: Jun. 7, 2006

(30) Foreign Application Priority Data
Jan. 23, 2003 (US) 60442030

(51) Int. Cl. A61K 48/00; C12N 15/86

(52) U.S. Cl. 424/93.2; 435/457; 977/804

(57) ABSTRACT

The invention includes methods for delivering therapeutic agents to a patient through administration of herpesvirus amplicon particles generated by a cell that stably express herpes simplex virus (HSV) immediate early 3 (IE3) gene.

### RELATED APPLICATIONS

This application claims the benefit of United States Provisional Patent Application U.S. Ser. No. 60/442,030, filed Jan. 23, 2003. The contents of this provisional patent application is hereby incorporated by reference in the present application in their entirety.

### STATEMENT REGARDING GOVERNMENT SUPPORT

The work described herein was funded, in part, by grants from the United States National Institutes of Health. The United States government may, therefore, have certain rights in the invention.

### FIELD OF THE INVENTION

The present invention relates to herpesvirus amplicon particles and methods of using them to, for example, deliver therapeutic agents to patients.

### BACKGROUND

Herpesviruses, such as herpes simplex virus (HSV), are DNA viruses capable of rapidly and efficiently infecting a wide variety of cell types (Leib and Olivo, BioEssays 15:547-554, 1993). These viruses can package material provided by way of amplicon plasmids or amplicon DNAs.

### SUMMARY

The present invention features compositions and methods for producing herpesvirus amplicon particles, including particles that integrate into the genome of a host cell. Presently known methods, while effective, can produce amplicon particles having low titers. We believe this results from the sub-optimal state of the HSV genome at the beginning of amplicon production, as the genome is without co-packaged viral regulators vhs and VP16. The vhs protein has an endoribonucleolytic activity that is important in the time-dependent progression of HSV gene expression and virion assembly, and VP16 is a strong transcriptional activator protein. Introducing these HSV proteins, as wild type or mutant proteins, into virus packaging cells during amplicon production enhances the number of viable viral particles. Moreover, the use of mutant forms of vhs in the production methods can result in higher virus particle number stocks that exhibit a diminished toxicity profile. While advantages that may be associated with various embodiments of the present invention are described further below, we note here that we expect the present methods to generate higher titer, safer vector stocks that can be used in HSV amplicon-based gene transfer methods. Moreover, although HSV amplicon particles can efficiently infect non-dividing cells and express transgenes therein, long-term expression in actively dividing cells has proven difficult. The amplicon genome, which exists episomally within nuclei of infected cells, is unable to replicate or segregate properly following cell division and therefore, is diluted over time from dividing cell populations. We combined the Tc1-like Sleeping Beauty (SB) transposon system with the amplicon to create a gene transfer vector that can integrate into the genomes of both dividing and non-dividing cell types. Vector integration within cells can extend the period of expression (e.g., expression of a therapeutic agent carried by an amplicon particle) when compared to cells transduced with more conventional HSV amplicons. The HSV amplicon systems described herein have a broader range of utility, as they can exhibit longer-term expression profiles.

Accordingly, the present invention features, inter alia, compositions and methods for generating herpesvirus amplicon particles. The compositions and the methods by which they can be made and used, are based, in part, on our realization that the relationship between VP16 and the virion host shut-off (VHS) protein could be exploited to limit herpesvirus-mediated vector toxicity. VP16 and VHS are tegument proteins that influence each other's activity; VP16 limits the ability of VHS to degrade RNA and inhibit host protein synthesis, and VHS can dampen the expression of VP16, which directs expression of potentially toxic immediate early (IE) genes (this interaction is further described below). Accordingly, the methods of the invention (at least those aimed at generating a viral amplicon particle) are based on a replication protocol that utilizes a virus (e.g., a herpes simplex virus such as HSV-1) deficient in VP16 (also known as α-trans inducing factor or αTIF) and/or VHS expression to function as a helper virus in amplicon vector packaging. By delivering VP16 and VHS in trans during the packaging process, there can be an improvement in, for example, amplicon titers or the amplicon to helper virus (A:H) titer ratio. Interestingly, VHS mutants deficient in mRNAse activity supported these trends as efficiently as the wild type protein. Thus, as described further below, the methods of generating a viral amplicon particle can be carried out using a variety of different viruses, including those deficient in wild-type VP16 and/or VHS function (whether deficient by virtue of omission or by mutation (e.g., a mutation that diminishes their activity)). Moreover, in some embodiments, herpesvirus amplicon particles can be produced following a single-round transfection-infection packaging procedure.

More specifically, the methods of generating a herpesvirus amplicon particle can be carried out by providing a cell permissive for herpesvirus (e.g. HSV) propagation; infecting the cell with a helper virus that has a diminished ability to express biologically active VP16, VHS, or both; transfecting the cell with a first plasmid that includes a herpesvirus (e.g., an HSV) origin of replication, a herpesvirus (e.g., an HSV) cleavage/packaging signal, and a heterologous (e.g., non-herpesvirus; non-HSV) transgene; and transfecting the cell with a second plasmid that includes a sequence that encodes a protein that is, or that is functionally equivalent to VP16 (in the event the helper virus has a diminished capacity to express biologically active VP16) or VHS (in the event the helper virus has a diminished capacity to express biologically active VHS). Optimally, the protein encoded by the second plasmid will bias virus propagation toward viruses that package amplicon DNA and away from the propagation of helper viruses. Optionally, where chromosomal integration is desired, the cell can be one that is transfected with a third plasmid that encodes a transposase (e.g., the transposase encoded by Sleeping Beauty) or a biologically active fragment or other mutant thereof. The infecting and transfecting steps can be carried out in the order given here, in a different order, or simultaneously (as can the steps of any other method of the invention in which cells are infected and/or transfected with more than one vector (here, we describe the vector is a plasmid, but those of ordinary skill in the art would understand that other types of vectors could be used in the methods of the invention as well).

Unless specifically noted herein, we use the terms “protein(s)” and “polypeptide(s)” interchangeably to refer to polymers of naturally or non-naturally occurring amino acid residues, whether glycosylated or not, and whether otherwise post-translationally modified or not.

In addition to various methods for making herpesvirus amplicon particles, the invention features methods for introducing nucleic acid sequences into cells (in vivo or in culture) using those particles. The nucleic acid sequences may be referred to herein as “transgenes”, and they can encode a variety of therapeutic polypeptides (or “agents”). In the event particles are introduced into cells in culture, the particle-containing cells can then be administered to patients. The cells administered may have been obtained initially from a patient and subsequently placed in culture; the administration can be of an autologous cell. However, the invention is not so limited. The cell can be any of a wide variety of types, so long as it is permissive for herpesvirus propagation. The cell must also supply certain elements lacking from the helper virus. For example, if an immediate early gene (e.g., IE3) is essentially missing from the helper virus (by virtue of a complete or partial deletion or other mutation that renders the gene product non-functional), that immediate early gene must be provided in (e.g., expressed by) the cell (whether a primary cell from a patient, from a cell line, or from another source). Moreover, the particles can be used in combination with (e.g., administered to a cell along with) a vector that expresses an enzyme (e.g., a transposase) that facilitates chromosomal integration of the transgene carried by the amplicon particle. Accordingly, the invention features cells bearing chromosomally integrated transgenes and kits containing those cells (or cells in which the transgene is carried episomally) or one or more of the materials used to generate the cells (e.g., one or more of the viral or plasmid vectors described herein and instructions for their use). The materials of the kits of the invention are preferably packaged in sterile form and with instructions for use.

Chromosomal integration can result in longer-term expression of the transgene. In either event (whether one generates cells in which gene expression is altered by episomally- or chromosomally-integrated nucleic acid sequences), the amplicon particles (or cells that contain them) can be administered to patients who have any of a wide variety of diseases or conditions. For example, they can be administered to a patient who has an infectious disease, cancer (or another cell proliferative disorder), a neurological deficit (including those in which neuron-specific proteins (e.g., neurotransmitters) are defective or underexpressed), a disease or condition that results from a genetic defect, or hearing loss. We describe the conditions amenable to treatment and the herpesvirus-based methods by which they can be treated in more detail after summarizing the methods for making the herpesvirus particles.

In any of the production or therapeutic methods described herein, the following additional limitations may apply. For example, the herpesvirus can be any of the more than 100 known species of herpesvirus, such as an alpha herpesvirus (e.g., a Varicella-Zoster virus, a pseudorabies virus, or a herpes simplex virus (e.g., type 1 or type 2 HSV) or an Epstein-Barr virus). Similarly, the methods can require sequences that encode an accessory protein, which can inhibit the expression of a gene in the cell. For example, the accessory protein can be a virion host shutoff (vhs or VHS) protein, and any of the methods of the invention that include expression of a VHS protein can employ, for example, an HSV-1 vhs protein, an HSV-2 vhs protein, an HSV-3 vhs protein, bovine herpesvirus 1 vhs protein, bovine herpesvirus 1.1 vhs protein, gallid herpes-virus 1 vhs protein, gallid herpesvirus 2 virion hsp, suid herpesvirus 1 vhs protein, baboon herpesvirus 2 vhs protein, pseudorabies vhs protein, cercopithecine herpesvirus 7 vhs protein, meleagrid herpesvirus 1 vhs protein, equine herpesvirus 1 vhs protein, or equine herpesvirus vhs protein). Any of these proteins can be operatively coupled to its native transcriptional control element(s) or to an artificial control element (i.e., a control element that does not normally regulate its expression in vivo).

As noted above (and elsewhere herein), the methods in which herpesvirus amplicon particles are generated can be carried out by transfecting a cell with a sequence encoding VP16 (this sequence can encode a full-length protein or a biologically active fragment or other mutant thereof), or a sequence that encodes a transcriptional activator that mimics VP16. The sequence encoding VP16 or a transcriptional activator that mimics VP16 can be introduced into packaging cells prior to the packaging components. The activation domain can be replaced with another regulatory protein so long as the signal that regulates the CAT/GRATATGARAT sequences (shown below) is retained. While “pre-loading” the packaging cells with VP16 is not essential, it can be done within the context of the present methods, and it can lead to an additional enhancement of amplicon particle titers. Moreover, the methods can be carried out with cells in which VP16, or a biologically active variant thereof, is stably expressed (methods to achieve stable expression are known in the art). Cells, including cells of any type that package amplicon particles, and that stably express VP16 are within the scope of the present invention. The cells can be those in which the VP16 protein is HSV1 VP16, HSV-2 VP16, bovine herpesvirus 1 VP16, bovine herpesvirus 1.1 VP16, gallid herpesvirus 1 VP16, gallid herpes-virus 2 VP16, meleagrid herpesvirus 1 VP16, or equine herpesvirus 4 VP16, or biologically active variants thereof.

VHS, or a biologically active variant thereof, can also be stably expressed so long as its expression can be suitably controlled. For example, one can control the expression of a sequence encoding VHS (or a biologically active fragment or other mutant thereof) by placing it in the context of a tetracycline, RU46, or ecdysone system. Such VHS-expressing plasmids are within the scope of the present invention. Similarly, the methods in which herpesvirus amplicon particles are generated by transfecting a cell with a sequence encoding VHS can be carried out with VHS (e.g., the VHS encoded by gene UL41) or with a mutant VHS, particularly one in which RNAse activity is reduced. Examples of VHS mutations that lead to abolished RNAse activity are the R27, Sc243, and M384 mutations described previously by Jones et al. (J. Virol. 69:4863-4871, 1995).

The methods of the invention can result in higher amplicon particle titers (e.g., 2-, 5-, 10- (or more) fold higher amplicon particle titers) and stocks that do not exhibit (or do not substantially exhibit) the pseudo-transduction phenomenon. These parameters can be evaluated by histochemistry (e.g., immunohistochemistry).

The therapeutic agent can be a protein or an RNA molecule (e.g., an antisense RNA molecule, a ribozyme, or a small interfering RNA (siRNA) that mediates RNA interference (RNAi)). In the event the therapeutic agent is a protein, the protein can be a receptor (e.g., a receptor for a growth factor or neurotransmitter), a signaling molecule (e.g., a growth factor or neurotransmitter), a transcription factor, a factor that promotes or inhibits apoptosis, a DNA replication factor, an enzyme, a structural protein, a neural protein (i.e., a protein expressed or differentially expressed in neurons), a heat shock protein, or a histone. Alternatively, or in addition, the therapeutic protein expressed can be an immunomodulatory protein (e.g., a cytokine, such as an interleukin, an interferon, or a chemokine, or a costimulatory molecule, such as a B7 molecule or CD40L), a tumor-specific antigen (e.g., PSA), or an antigen of an infectious agent (e.g., a virus such as a human immunodeficiency virus (in which case the antigen can be, for example, a Gag, Pol, or Env protein, or a variant thereof), a herpesvirus, a papillomavirus, an influenza virus, or Ebola virus, a bacterium (e.g. an Escherichia (e.g., E. coli) Staphylococcus, Campylobacter (e.g., C. jejuni), Listeria (e.g., L. monocytogenes), Salmonella, Shigella, or Bacillus (e.g., Bacillus anthracis)), or a parasite. Other therapeutic agents include immunomodulatory proteins, tumor-specific antigens, and the antigens of infectious agents (including antigens associated with the infectious agents described above). It will be apparent to one of ordinary skill in the art which therapeutic agents can be expressed to generate amplicon particles and cells useful for treating a given condition. For example, one would select an antigen expressed by HIV (e.g., gp120 and/or Gag-Pol) to treat a patient who is infected, or who may become infected, with HIV; one would select a prion protein to treat a patient who has, or who is at risk of developing, CJD; one would select a neurotransmitter or other neural protein to treat a patient who has a neurological deficit; and so forth. The therapeutic agents administered by the methods of the present invention can also include known therapeutic peptides or peptidomimetics and those under development (e.g., T-20 and T-1249, under development by Trimeris (Durham, N.C.)).

In other embodiments, the invention features methods that include isolating the herpesvirus amplicon particles produced by host cells treated as described herein, the amplicon particles including a transgene (that expresses a therapeutic agent) (see the PCT application published under number WO 01/89304, which, for the purpose of at least U.S. patent prosecution, is incorporated herein by reference in its entirety). Briefly, particle-containing cells are lysed, cellular debris may be cleared or reduced, and the particle-containing fraction is applied to a sucrose density gradient (particles come to reside at the interface). Purification can also be achieved by affinity chromatography. For example, one can immobilize an antibody or a fragment thereof (e.g., a single chain antibody that may be humanized) that recognizes a protein on the herpes virion (e.g., an Env protein). The antibody can be immobilized on a column or other solid support. Once immobilized, the antibody can be exposed to a sample containing amplicon particles under conditions in which the antibody can specifically bind the particles. After the remainder of the sample is washed away, the antibody-particle interaction can be broken (e.g., the complex can be cleaved with a protease (e.g., an endopeptidase, a viral protease, or a combination thereof). Preferably, no protein is cleaved from the virion/amplicon particle.

In other embodiments, as mentioned above, the invention features methods in which a transgene of a herpesvirus amplicon particle (e.g., an HSV amplicon particle) is integrated into the chromosomes of dividing and non-dividing cells. This integrating form of the amplicon can be produced using the VHS and VP16-based methods of packaging described above using either helper virus or helper virus-free technology. The conventional amplicon genome is maintained as an episome and is not mitotically maintained during cell division. However, vectors made by the methods described herein can be used to transfer transgenes from parent cells to daughter cells. The methods can be carried out by combining a transposon-encoding system (e.g., the Tc1-like Sleeping Beauty (SB) transposon system) with the amplicon particle produced by the methods described herein. When cells contain both an enzyme that mediates chromosomal integration and a corresponding amplicon particle bearing a heterologous transgene flanked by the transpose recognition sequences, the transgene can integrate into the genomes of both mitotically active and post-mitotic cell types.

In addition, the invention features kits containing one or more of the herpesvirus amplicon particles described herein; one of more cells or cell types containing them; or one or more of the components useful in generating either the particles or the cells. For example, a kit can include a helper virus and an amplicon plasmid. Optionally, the kit can also contain cells, which can be stably transfected. Optionally, the kit can include instructions for use, and any of the kits that contain one or more components of the amplicon system (e.g., the components enumerated above) can also contain a vector that encodes an enzyme that mediates integration of the transgene carried by the amplicon particle into the genome of a host cell.

The particles generated by the methods of the invention, cells that contain those particles, and the components used to generate them are also within the scope of the invention. The particles and cells that come within the scope of the invention include any of those made using the methods described herein. The cell can be virtually any differentiated cell or a precursor thereof. For example, the cell can be a neuron, a blood cell, a hepatocyte, a keratinocyte, a melanocyte, a neuron, a glial cell, an endocrine cell, an epithelial cell, a muscle cell, a prostate cell, or a testicular cell. The cell can also be a malignant cell (including any of those that arise from the differentiated cells just listed; e.g., a neuroblastoma, a lymphoma or leukemia cell, a hepatocarcinoma cell etc.). Alternatively, or in addition, the cell can be any cell that is infected with an infectious agent (including a virus, a bacterium, a parasite, or a prion including, but not limited to, those types described herein).

The herpesvirus amplicon particles, regardless of the precise method by which they are made, can contain one or more genes encoding one or more therapeutic proteins (full-length or biologically active or therapeutically effective fragments or mutants thereof), and they can be used to transduce cells, including those that contain an infectious agent. The term “infectious agent,” as used herein, encompasses viruses, bacteria (including both eubacteria and archaea), fungi, mycobacteria, mycoplasmas, protoctistans, parasites, and prions unless a specific exception is explicitly noted in the description below; a cell that contains an infectious agent may be referred to herein as an infected cell (and may be a cell from a human, cow, sheep, pig, goat, horse, dog, cat, mouse (or other rodent) or another animal (including non-human primates)). Thus, while the compositions and methods described herein can be administered to (or applied to) humans, they can also be administered to (or applied to) domesticated animals, laboratory animals, or livestock. As noted above, the patient can have any one of a wide variety of infectious diseases, including those associated with non-conventional infectious agents, such as prions (e.g., a transmissible spongiform encephalopathy (TSE) such as Creutzfeld-Jacob disease (CJD) or Gertsmann-Straussler-Scheinker syndrome (GSS) in humans) and/or any one of a wide variety of cancers (including chronic lymphocytic leukemia, other cancers in which blood cells become malignant, and lymphomas (e.g. Hodgkin's lymphoma or non-Hodgkin's type lymphomas), a melanoma, a glioblastoma, an astrocytoma, a pancreatic cancer, a cancer of the reproductive system, a cancer of the endocrine system, a neuroblastoma, a breast cancer, a colorectal cancer, a stomach cancer, a cancer of the throat or within or around the mouth, a lung cancer, or a bladder cancer). Other conditions amenable to treatment include neurological disorders (e.g., Alzheimer's Disease, Huntington's Disease, and Parkinson's Disease; additional exemplary conditions are disclosed below) and disorders that result in partial or complete loss of hearing (including loss with age).

HSV amplicon particles have been used to express neuroprotective or neuroregenerative factors at high levels in various disease settings. Disease targets related to hearing loss have proven especially amenable to HSV-directed gene transfer. In the context of age-related hearing loss (presbycusis) and ototoxic drug-induced hearing loss (e.g., hearing loss following administration of aminoglycosides or cisplatin), HSV amplicon particles that express the neurotrophic factor NT-3 have provided protection against spiral ganglion neuron (SGN) degeneration. Accordingly, one can treat a patient who has, or who is likely to have, some hearing loss by administering herpesvirus amplicon particles, made by the methods described herein, that express neurotrophic factors before, during, or after a patient has been exposed to an agent (e.g., a chemotherapeutic agent) that adversely affects cells within the auditory system (e.g., SGNs).

Without limiting the amplicon particles of the invention to those that have any particular characteristics, preliminary proteomic data suggests that the particles made by the methods described herein are distinct from those made by other systems.

The therapeutic protein expressed by the particles can be an immunostimulatory protein and may be a neoantigen (e.g., a tumor-specific antigen, such as prostate-specific antigen (PSA)). For example, the immunostimulatory protein can be an antigen associated with (e.g., expressed by) an infectious agent such as a prion protein or a non-infectious mutant or fragment thereof. The immunostimulatory protein can also be a particular viral antigen or an antigenic fragment thereof (e.g. the immunostimulatory protein can be tat, nef, gag/pol, vp, or env from an immunodeficiency virus such as HIV-1 or HIV-2) or a particular bacterial, mycobacterial, parasitic, or other infectious organismal antigen or an antigenic fragment thereof. For example, the therapeutic protein can be a portion of Prp^{c} (the non-infectious normal cellular prion protein) (e.g., residues 76-112; 134-160; 150-177; or 198-228 of SEQ ID NO:1; additional prion sequences are known by, and available to, those of ordinary skill in the art and can also be used as described herein). Alternatively, or in addition, the hf-HSV particles of the invention can be used to express single-chain variable regions of antibodies (scFv), including those specific to Prp^{sc} (infectious prion agents). Similarly, single chain antibodies (that can be humanized by methods known in the art) that are directed against pathogenic antigens can be administered to patients who have been, or who may be, infected with or exposed to those agents. Expression of single-chain variable regions can be used to treat other conditions (e.g., cancer and neurological disorders) as well. For example, variable regions that specifically bind Aβ and α-synuclein can be used to treat patients who have, or who may develop, Alzheimer's Disease or Parlinson's Disease, respectively. As noted elsewhere, the transgene included in the amplicon particle may encode an antisense oligonucleotide, an siRNA, or an RNAi.

In one embodiment, an affected cell (e.g., an infected cell, a malignant cell, or one affected by neurological disease) is transduced (in vivo or ex vivo) with an HSV amplicon particle that encodes an immunostimulatory protein (i.e., any protein or peptide that, when expressed by a target cell, induces or enhances an immune response to that cell). For example, a patient who has cancer can be treated with an HSV amplicon particle (or a cell within which it is contained) that expresses an antigen and a polypeptide that acts as a general stimulator of the immune system or a specific protein, such as a tumor-specific antigen (e.g., prostate-specific antigen (PSA)) (these particles and cells can be those made by the methods described herein). Similarly, a patient who has an infectious disease can be treated with an HSV amplicon particle (or a cell within which it is contained) that expresses an antigen and a polypeptide that acts as a general stimulator of the immune system or a specific antigen associated with (i.e., expressed by) the infectious agent (here again, the patients that are treated for an infectious disease can be treated with particles or cells made by the methods described herein). Polypeptides that act as general stimulators of the immune system include cytokines, including chemotactic cytokines (also known as chemokines) and interleukins, adhesion molecules (e.g., I-CAM) and costimulatory factors necessary for activation of B cells or T cells.

More generally, the methods of the invention including treating patients (such as those described above) by (a) providing an HSV amplicon particle that includes at least one transgene that encodes a therapeutic product and (b) exposing cells of the patient (e.g., pathogen-infected cells, malignant cells, or neural or pre-neural cells) to the herpesvirus amplicon particles under conditions effective for infective transformation of the cells. The therapeutic transgene product is expressed in the cells (e.g., in vivo) and thereby delivers a therapeutically effective amount of the therapeutic product to the patient. Physicians and others of ordinary skill in the art are well able to determine whether an agent is therapeutically effective. They can, for example, observe an improvement in an objective sign of disease (e.g., an improvement in cognitive skills, motor skills, memory, platelet count, reduction of fever, or reduction of tumor size). An agent is also therapeutically effective when a patient reports an improvement in a subjective symptom (e.g. less fatigue, feeling “better”).

Gene therapy vectors based on the herpes simplex virus have a number of features that make them advantageous in clinical therapies. Such vectors, including the amplicon particles described herein, can, in various embodiments, have one or more of the following attributes: they can exhibit a broad cellular tropism, they can have the capacity to package large amounts of genetic material (and thus can be used to express multiple genes or gene sequences), they can have a high transduction efficiency, and they can be maintained episomally, which makes them less prone to insertional mutagenesis (Geller and Breakefield, Science 241:1667-1669, 1988; Spaete and Frenkel, Cell 30:305-310, 1982; Federoff et al., Proc. Natl. Acad. Sci. USA 89:1636-1640, 1992; Federoff in Cells: A Laboratory Manual, Spector et al., Eds., Cold Spring Harbor Press, Cold Spring Harbor, N.Y., 1997; Frenkel et al., in Eucaryotic Viral Vectors, Gluzman, Ed., Cold Spring Harbor Press, Cold Spring Harbor, N.Y., 1982). In addition to infecting many different types of cells, HSV vectors can transduce non-replicating or slowly replicating cells, which has therapeutic advantages. For example, freshly isolated cells can be transduced in tissue culture, where conditions may not be conducive to cell replication. The ability of HSV vectors to infect non-replicating or poorly replicating cells also means that cells (such as tumor cells) that have been irradiated can still be successfully treated with HSV vectors. The amplicon particles described herein may also be more amenable to large-scale production of safe amplicon stocks.

The transduction procedure can also be carried out fairly quickly; freshly harvested human tumors have been successfully transduced within about 20 minutes. As a result, cells (such a tumor cells) can be removed from a patient, treated, and readministered to the patient in the course of a single operative procedure (one would readminister tumor cells following transduction with, for example, an immunostimulatory agent (HSV vectors encoding immunomodulatory proteins and cells transduced with such vectors can confer specific anti-tumor immunity that protects against tumor growth in vivo)).

On the other hand, it is inherently difficult to manipulate a large viral genome (150 kb), and HSV-encoded regulatory and structural viral proteins may be toxic (Frenkel et al., Gene Ther. 1 Suppl. 1:S40-46, 1994). Efforts to bring this vector system into the clinical arena to treat neurodegenerative disease have been hampered by potential cytotoxicites that are associated with traditional methods of virus packaging. This problem involves the co-packaging of helper virus that encodes cytotoxic and immunogenic viral proteins. Newer methods of packaging have been developed that result in helper virus-free amplicon stocks (Fraefel et al., J. Virol. 70:7190-7197, 1996; Stavropoulos and Strathdee, J. Virol. 72:7137-7143, 1998; see also U.S. Pat. Nos. 5,851,826 and 5,998,208). Stocks prepared by these methods, however, are typically low titer (<10⁵ expression units/ml), allowing for only modest scale experimentation, primarily in vitro. Such low titers make large animal studies difficult, if not impossible.

Other features and advantages of the invention will be apparent from the following detailed description, and from the claims.

### BRIEF DESCRIPTION OF THE DRAWINGS

FIGS. 1A and 1B are schematic representations of virus lineage and a restricted replication assay protocol, respectively. FIG. 1A illustrates derivations of the helper deletion mutant viruses used to package amplicon vectors. The D30EBA virus has been deleted of both copies of the IE3 gene and, like the other deletion mutants, must be propagated on the BHK-based, IE3-expressing stable RR1 packaging cell line. The VP16 or vhs loci were mutated by linker insertion or by homologous recombination using the vhs reading frame interrupted by a CMVlacZ transcription unit as described (Johnson et al., J. Virol. 68:6347-6362, 1994). FIG. 1B illustrates the IE3 defective helper virus D30EBA, which is complemented by the RR1 cell line and is capable of generating helper virus and amplicon-containing virions in cells receiving the amplicon plasmid (black). Co-expression of either wild type VP16 or VHS (not shown) complements additional mutations harbored in the helper viruses described above and permits efficient amplicon and helper virus generation in cells receiving the expression plasmid (gray).

FIGS. 2A and 2B are bar graphs of data concerning VP16-mediated enhancement of amplicon titers (amplicon DNA recovered (ng) and amplicon titer (TU/ml×10,000), respectively). The data shown in FIG. 2A was collected following co-transfection of a VP16 expression plasmid with the amplicon reporter plasmid prior to super-infection, which augments the production of infectious amplicon-containing virus. Quantitative PCR using LacZ-specific primers was performed on DNA extracted from Vero monolayers transduced with 14HΔ3±VP16 packaged amplicon stocks (n=3). FIG. 2B illustrates the finding that VP16 delivery enhances amplicon titers independent of binding to TAATGARAT (SEQ ID NO:2) elements. Amplicon plasmids either containing (HSVlac) or deleted (CMVlac) of the VP16 binding element were packaged using the 14HΔ3 helper virus, either with (filled bars) or without (open bars) the co-transfected VP16 expression vector.

FIG. 3 is a bar graph generated following experiments to assess cellular toxicity (% survival). Vectors packaged by restricted replication exhibit reduced neurotoxicity. HSVlucCMVegfp vector stocks packaged with helper viruses either in the absence or presence of VP16 added in trans (+VP16) were used to transduce cortical neurons cultured in Neurobasal media with B27® supplement at an MOI of 2.0. Viability was assessed by the MTS method at 24 (open bars) and 72 hours (filled bars) post-infection. Values represent the average survival from six infected wells relative to un-infected control cultures. Student t-tests for 24 (+=p<0.01) and 72 hour (**=p<0.001; ***=p<0.0001) time points were determined comparing test samples against the D30EBA-packaged virus.

FIG. 4 is a bar graph generated following experiment to assess viability. VHS mRNAse activity promotes toxicity in transduced neuronal cultures. HSVlucCMVegfp vector stocks packaged with D30EBA or the triple mutant 14HΔ3vhsZ in the absence or presence of wild-type VP16 or various VHS expression constructs added in trans were used to transduce cortical neurons cultured in Neurobasal media with B27® supplement at an MOI of 2.0. Survival was measured at 24 (open bars) and 72 hours (filled bars) post-transduction using the MTS assay as described. Data presented represent an average±standard deviation from six infected wells and viability is expressed as a relative percent of the uninfected control wells. Student t-tests were performed at the 72 hour time point (**=p<0.005, ***=p<0.001) comparing test samples against the D30EBA-packaged virus.

FIG. 5 is a Table of essential HSV-1 genes.

FIGS. 6A and 6B are schematic representations of suitable amplicon vectors. FIG. 6A represents the empty amplicon vector pHSVlac, which includes the HSV-1 a segment (cleavage/packaging or pac signal), the HSV-1 c region (origin of replication), an ampicillin resistance marker, and an E. coli lacZ marker under the control of HSV IE4 promoter and SV40 polyadenylation signal. FIG. 6B represents insertion of a transgene in a site (BamHI) adjacent to the HSV-1 a segment, forming pHSVlac/trans.

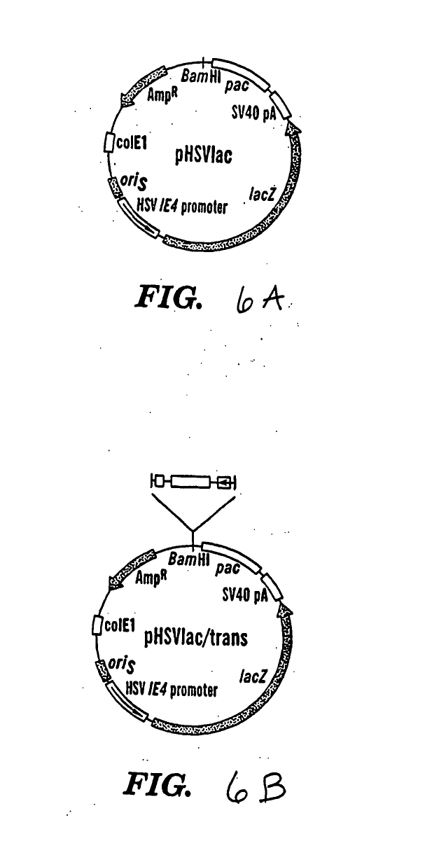

FIG. 7 is a bar graph demonstrating integration of HSV amplicon-delivered Sleepting Beauty/T-βgeo transposon in BHK cells. Monolayers of BHK cells were left untreated or were transduced with 5×10⁴ virions of HSVsb alone, HSVT-βgeo alone, or HSVT-βgeo plus HSVsb. Three days later, cultures were placed under G418 selection, which was continued for two weeks to allow for colony growth. Resultant G418-resistant colonies were stained with X-gal and enumerated. Co-transduction of HSVT-βgeo and HSVsb led to a significant enhancement of drug-resistant colony formation, suggesting integration has occurred in the mitotically active BHK cells. The “*” indicates a statistically significant difference between HSVT-βgeo alone and HSVT-βgeo plus HSVsb treatment (p<0.05) (see also Example 15).

FIGS. 8A-8C are bar graphs demonstrating that co-transduction of primary neuronal cultures with HSVT-βgeo and HSVsb results in enhanced gene expression and high retention of transgenon DNA. Primary neuronal cultures established from E15 mouse embryos were transduced with HSVsb and/or HSVT-βgeo and analyzed at Days 4 or 9 post-transduction by enumeration of LacZ-positive cells (FIG. 8A), β-galactosidase activity (FIG. 8B) and quantitation of retained transgenon DNA sequences (FIG. 8C). The “*” indicates a statistically significant difference between HSVT-βgeo alone and HSVT-βgeo plus HSVsb combination group (p<0.05).

FIG. 9 is a schematic representation of a construct of the invention within the genome of a host cell. These constructs can be packaged into HSV particles using the methods described herein. Primary neuronal cultures established from E15 mouse embryos were transduced with HSVsb and HSVT-βgeo and high molecular weight DNA harvested on Day 9 post-transduction. Inverse PCR was performed to determine novel flanking sequences of the integrated transgenon using a series of nested primers. Amplified products were isolated, cloned, and sequenced. Novel mouse-derived flanking sequences are shown.

FIGS. 10A-10C are bar graphs of various parameters measured after transduction with HSVsb and/or HSVT-βgeo. HSVsb and/or HSVT-βgeo were administered stereotactically to the striata of C57BL/6 mice and animals were sacrificed at 7, 21, and 90 days post-transduction. HSVPrPUC amplicon virions were included in the HSVsb only and HSVT-βgeo only groups to normalize viral particle input. Tissue blocks consisting of the striatal injection site were excised, homogenized, and analyzed initially for β-galactosidase reporter gene expression by the Galacto-Lite assay (FIG. 10A). Total genomic DNA was purified from these lysates and subjected to real-time quantitative PCR to detect either transgenon sequences (FIG. 10B) or sequences specific to the Sleeping Beauty—expressing amplicon vector (FIG. 10C). The “*” indicates a statistically significant difference between HSVT-βgeo alone and HSVT-βgeo plus HSVsb treatments (p<0.05).

### DETAILED DESCRIPTION

Given their natural ability to infect post-mitotic neurons, to undergo retrograde transport, and to accept transcription units ≦approximately 130 kb, HSV-based vectors are well-suited for gene transfer, including gene transfer into the CNS. The conventional amplicon packaging system utilizes a replication-defective helper virus deleted in one or more immediate early (IE) regulatory genes to supply in trans the machinery required to direct packaging of a plasmid-based expression vector. The amplicon plasmid contains the HSV origin of replication and accessory elements necessary for DNA replication and inclusion into infectious HSV particles. These vectors also support the constitutive or regulated expression of one or more open reading frames through the use of viral promoters, cell type-specific promoters, and regulated promoter elements whose expression is dependent on ligand-receptor interactions (Lu and Federoff, Hum. Gene Ther. 6:419-428, 1995; Ho et al., Brain Res. Mol Brain Res. 41:200-209, 1996). While this platform is capable of producing vector titers several log orders above those generated using cosmid or BAC-based helper-free systems, there may be some toxicity imparted by residual IE gene expression from the contaminating helper virus (Johnson et al., J. Virol. 66:2952-2065, 1992).

In the natural course of infection, the coordinated action of preformed viral proteins as well as the actions of immediate-early (IE), early, and late genes expressed de novo from the viral genome following infection are required to direct efficient viral replication. The α-trans inducing factor (αTIF or VP16) is an integral tegument protein required for encapsidation of viral DNA and egress beyond the peri-nuclear space. Consequently, deletion of VP16 from the viral genome arrests the production of infectious particles (Weinheimer et al., J. Virol. 66:258-269, 1992). With the cooperation of cellular factors including Oct-1/POU and HCF, VP16 also directs robust immediate-early gene expression by facilitating the assembly of pre-initiation complexes at TAATGARAT (SEQ ID NO:2) cis regulatory elements contained within immediate-early promoter regions (Rhys et al., J. Virol. 63:2798-2812, 1989). The potent carboxy-terminal domain of VP16 supports robust gene expression by recruiting transcription-associated factors with histone acetyl-transferase (HAT) activity, thereby promoting transcription (Utley et al., Nature 394:498-502, 1998). The HSV-1 encoded, VP16-responsive immediate-early genes IE1 (ICPO), IE2 (ICP27) and IE4 (ICP22) are each capable of producing significant toxicity when expressed in cultured cell lines (Johnson et al., J. Virol. 68:6347-6362, 1994). Substitution of wild-type VP16 with insertion or deletion mutants attenuates the generation of high-titer progeny virus, a phenotype that can be reversed either by supplying wild-type VP16 in trans (Bowers et al., Gene Ther. 8:111-120, 2001), or by treatment with hexamethyl-bis-acetamide (HMBA) (Johnson et al., J. Virol. 68:6347-6362, 1994). Not surprisingly, the IE 3−/− VP16ⁱⁿ double mutant, 14HΔ3, exhibited reduced toxicity compared to its parental virus. Therefore, achieving balance between the efficient production of infectious progeny virus and VP16-regulated toxicity presents a challenge in incorporating such mutations into helper-based HSV-1 packaging methods.

The virion host shutoff (VHS) protein is contained within the tegument of the mature HSV particle and, like VP16, is delivered at the time of infection. In addition to imparting structural stability to the viral particle (Bowers et al., Gene Ther. 8:111-120, 2001), VHS also encodes an mRNAse function that catalyzes the rapid degradation of mRNA and the subsequent shutoff of host protein synthesis. In fact, VHS has been proposed as a potential suicide vector for use in cancer therapy given its observed in vitro cytotoxic potential (Glenn and Chatterjee, Cancer Gene Ther. 8:566-573, 2001). VHS also dampens VP16-mediated gene expression allowing for progression through the viral life cycle while reducing the accumulation of toxic immediate-early gene products. Conversely, VP16 (through its association with amino acids 238-344 of VHS) is capable of limiting VHS mRNAse activity (Schmelter et al., J. Virol. 70:2124-2131, 1996; Smibert et al., J. Virol. 68:2339-2346, 1994). Consistent with these findings, cells infected with a VP16-null mutant undergo translational arrest and apoptosis secondary to unopposed VHS activity, an effect reversed by deletion of the vhs gene (Lam et al., EMBO J. 15:2575-2581, 1996). VHS mutations that abolish RNAse activity are the R27, Sc243, and M384 mutations described previously by Jones et al. (J. Virol. 69:4863-4871, 1995). A strategy has been devised based on the complex reciprocal relationship between VP16 and VHS to limit HSV-1 amplicon mediated vector toxicity.

To understand the influence of VP16 and VHS on neuron survival following infection using helper virus-packaged amplicon stocks, a restricted replication protocol was developed that utilized HSV-1 viruses deficient in VP16 and/or VHS expression to function as helper virus in amplicon vector packaging. By delivering VP16 and VHS in trans during the packaging process, improvements were demonstrated in both amplicon titers and the amplicon to helper virus (A:H) titer ratios. Interestingly, VHS mutants deficient in mRNAse activity supported these trends as efficiently as the wild-type protein. Addition of wild-type VP16 and mutant VHS in trans to an amplicon packaging protocol, which utilized helper viruses harboring genomic mutations in both VP16 and VHS, markedly reduced toxicity on primary cortical neuronal cultures as compared to controls. These data support a neurotoxic role for VHS in the setting of amplicon infection and demonstrate that replacement of mutant for wild-type VHS improves the toxicity profile of HSV-1 amplicon and other herpes-based gene delivery systems.

In packaging systems like the ones described here, which employ helper viruses, amplicon plasmids rely on the helper virus function to provide the replication machinery and structural proteins necessary for packaging amplicon plasmid DNA into viral particles. Helper packaging function is usually provided by a replication-defective virus that lacks an essential viral regulatory gene. The final product of helper virus-based packaging contains a mixture of varying proportions of helper and amplicon virions.

The core of the herpesvirus particle is formed from a variety of structural genes that create the capsid matrix. It is necessary to have those genes for matrix formation present in a susceptible cell used to prepare particles. It can be beneficial for the necessary envelope proteins also to be expressed. In addition, there are a number of other proteins present on the surface of a herpesvirus particle. Some of these proteins help mediate viral entry into certain cells, and as this is known to those of ordinary skill in the art, one would know to alter the sequences expressed by the viral particle in order to alter the cell type the viral particle infects or improve the efficiency with which the particle infects a natural cellular target. Thus, the inclusion or exclusion of the functional genes encoding proteins that mediate viral entry into cells will depend upon the particular use of the particle.

In addition to a packaging vector, the herpesvirus amplicon systems described herein include an amplicon plasmid. The amplicon plasmid contains a herpesvirus cleavage/packaging site containing sequence, an origin of DNA replication (ori) that is recognized by the herpesvirus DNA replication proteins and enzymes, and a transgene of interest (e.g., a nucleic acid sequence that encodes a therapeutically effective protein). For example, the amplicon plasmid can contain at least one heterologous DNA sequence that is operatively linked to a promoter sequence (we discuss promoter and other regulatory sequences further below). More specifically, the amplicon plasmid can contain one or more of the following elements: (1) an HSV-derived origin of DNA replication (ori) and packaging sequence (“a” sequence); (2) a transcription unit driven typically, but not necessarily, by the HSV-1 immediate early (IE) 4/5 promoter followed by a gene (or genes or fragments thereof) and an SV-40 polyadenylation site; and (3) a bacterial origin of replication and an antibiotic resistance gene for propagation in E. coli (Frenkel, supra; Spaete and Frenkel, Cell 30:295-304, 1982). As noted elsewhere, the transgene could encode an siRNA (that is, small, interfering RNA); it does not have to encode protein.

Methods for Generating Herpesvirus Amplicon Particles

Generally, the methods of the invention are carried out by infecting and transfecting a host cell with several vectors and then isolating viral (e.g., HSV) amplicon particles produced by the host cell (while the language used herein may commonly refer to a cell, it will be understood by those of ordinary skill in the art that the methods can be practiced using populations (whether substantially pure or not) of cells or cell types, examples of which are provided elsewhere in our description). The method for producing a herpesvirus amplicon particle can be carried out, for example, by introducing, into a host cell (by, for example, infecting): a helper virus that is unable to express at least one protein, wherein the at least one protein is VP16 or a VHS protein; a first plasmid comprising an HSV origin of replication, an HSV cleavage/packaging signal, and a heterologous transgene; and a second plasmid comprising a sequence that encodes a protein that is, or is functionally equivalent to, the protein the helper virus fails to express. This protein complements the mutation(s) encoded by the helper virus and biases virus propagation toward viruses that package amplicon DNA and away from the propagation of helper virus. One can then isolate or purify (although absolute purity is not required) the herpesvirus amplicon particles produced by the host cell. To further enhance the use of the amplicon particles, the resulting stock can also be concentrated, which affords a stock of isolated HSV amplicon particles at a concentration of at least about 1×10⁷ particles per milliliter.

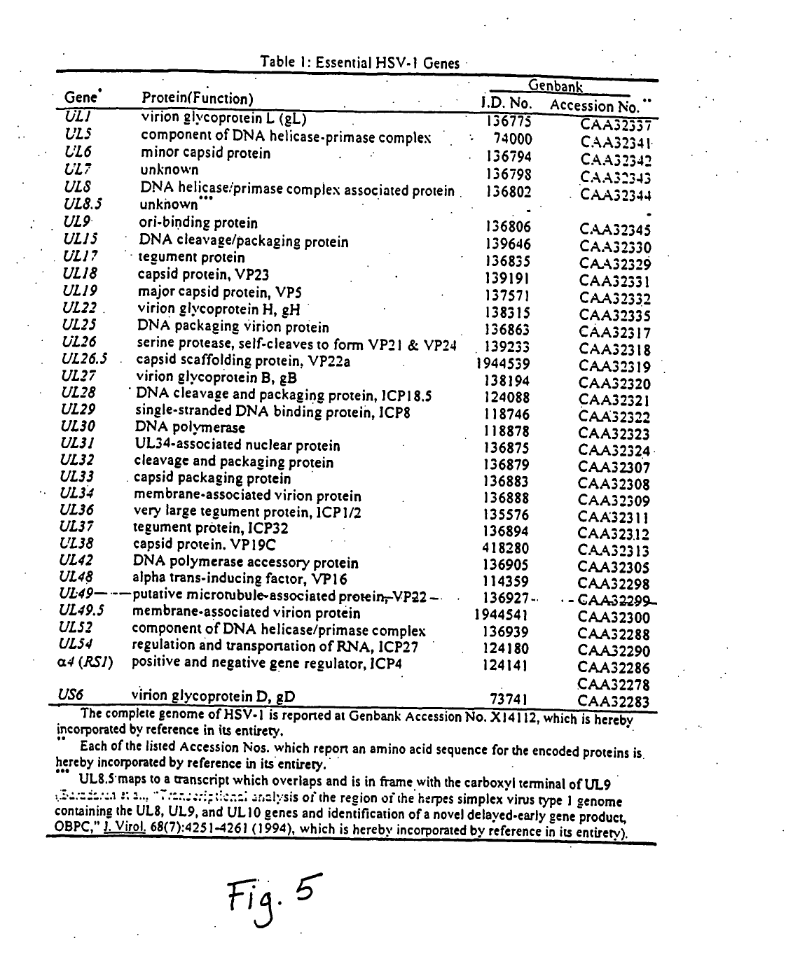

By “essential HSV genes” or by “all of the required HSV structural proteins”, it is intended that the one or more vectors (e.g., the infective helper virus) will include all of the genes that encode polypeptides that are necessary for replication of the amplicon vector and for structural assembly of the amplicon particles (the genes can have naturally occurring sequences or they can include mutations that do not substantially reduce their biological activity). Thus, in the absence of such genes, the amplicon vector is not properly replicated and packaged within a capsid to form an amplicon particle capable of adsorption. Such “essential HSV genes” and “required HSV structural proteins” have previously been reported in review articles by Roizman (Proc. Natl. Acad. Sci. USA 11:307-1 13, 1996; Acta Viroloeica 43:75-80, 1999; both of which are incorporated herein by reference). Another source for identifying such essential genes is available at the Internet site operated by the Los Alamos National Laboratory, Bioscience Division, which reports the entire HSV-1 genome and includes a table identifying the essential HSV-1 genes. The genes currently identified as essential are listed in the Table provided as FIG. 5.

The HSV cleavage/packaging signal can be any cleavage/packaging that packages the vector into a particle that is capable of adsorbing to a cell (the cell being the target for transformation). A suitable packaging signal is the HSV-1 “a” segment located at approximately nucleotides 127-1132 of the a sequence of the HSV-1 virus or its equivalent (Davison et al, J. Gen. Virol. 55:315-331, 1981).

The HSV origin of replication can be any origin of replication that allows for replication of the amplicon vector in the host cell that is to be used for replication and packaging of the vector into HSV amplicon particles. A suitable origin of replication is the HSV-I “c” region, which contains the HSV-I ori segment located at approximately nucleotides 47-1066 of the HSV-I virus or its equivalent (McGeogh et al., Nucl. Acids Res. 14:1727-1745, 1986). Origin of replication signals from other related viruses (e.g., HSV-2 and other herpesviruses, including those listed above) can also be used.

The amplicon plasmids can be prepared (in accordance with the requirements set out herein) by methods known in the art of molecular biology. Empty amplicon vectors can be modified by introducing, at an appropriate restriction site within the vector, a complete transgene (including coding and regulatory sequences). Alternatively, one can assemble only a coding sequence and ligate that sequence into an empty amplicon vector or one that already contains appropriate regulatory sequences (promoter, enhancer, polyadenylation signal, transcription terminator, etc.) positioned on either side of the coding sequence. Alternatively, when using the pHSVlac vector, the LacZ sequence can be excised using appropriate restriction enzymes and replaced with a coding sequence for the transgene. Conditions appropriate for restriction enzyme digests and DNA ligase reactions are well known in the art (see, e.g., Sambrook et al., Molecular Cloning: A Laboratory Manual, Cold Spring Laboratory, Cold Spring Harbor, N.Y. (1989); Ausubel et al. (Eds.), Current Protocols in Molecular Biology, John Wiley & Sons, New York, N.Y., 1999 and preceding editions; and U.S. Pat. No. 4,237,224).

The amplicon systems featured in these methods and others described herein can all be modified so that the transgene carried by the amplicon plasmid is inserted into the genome of the host cell. These integrating amplicons, as is the case with conventional episomal versions of the amplicon, can be packaged into virus particles using the methods described in this invention. Accordingly, the methods described herein can each include an additional step of introducing, into the host cell, a vector (which can be, but is not necessarily, a plasmid) that encodes an enzyme that mediates insertion of the transgene into the genome (this vector may be referred to herein as “an integration vector”). The integration vector can be applied to a host cell in vivo or in culture at the same time that one or more of the components of the amplicon system (e.g., the packaging vector or amplicon plasmid) are administered to the host cell. The enzyme encoded by the integration vector can be a transposase, such as that encoded by Sleeping Beauty or a biologically active fragment or mutant thereof (i.e., a fragment or mutant of the Sleeping Beauty sequence that facilitates integration of the transgene into the genome at a rate or to an extent that is comparable to that achieved when wild type Sleeping Beauty is used). As this system represents a fundamental advance over those in which the amplicon plasmid is maintained outside the genome (and is therefore “diluted out” as cells divide), it has broad application. Methods in which an integration vector is used in the context of an amplicon system, particularly including the hf-HSV systems described herein, can be carried out to treat patients with a wide variety of diseases or disorders (here, as in the methods described above, a “patient” is not limited to a human patient but can be any other type of mammal). For example, the patient can have cancer, an infectious disease, a neurological disease, or be suffering from a neuronal deficit that leads to sensory impairment, such as loss of hearing. Any of the specific types of cancer, infectious diseases, or neurological diseases set out herein can be treated. In addition, one can further modify the amplicon system to improve the safety of treatments in which an integration vector is administered. Frequent transposition events may lead to mutagenesis of the host genome and, possibly, even to proto-oncogene activation (although there is no evidence that this will occur or is likely to occur; we are speculating that the amplicon might enhance the frequency of such events, as 10-15 copies of the transgenon are present within a single virion). To regulate the transposase component of the system more tightly, one could, for example, incorporate the Sleeping Beauty protein into the virion in the form of a fusion with an HSV tegument protein. Alternatively, one could effect exogenous application of transposase protein with the transgenon-containing amplicon vector. Both approaches would prevent continued synthesis of Sleeping Beauty and thus, obviate additional catalysis of transposition. In yet another strategy, one could incorporate protein instability sequences into the open reading frame to limit transposase half-life. As illustrated in the examples provided below, the transposon in the integration vector should be compatible with sequences flanking the transgene in the amplicon plasmid. For example, where the transposon is of the Sleeping Beauty system, the amplicon vector can include a transgene (for integration) flanked by the Sleeping Beauty terminal repeats. Integrating forms of the HSV amplicon vector platform have been described previously. One form consists of an HSV amplicon backbone and adeno-associated virus (AAV) sequences required for integration (Costantini et al., Hum. Gene Ther. 10:2481-2494, 1999).

The amplicon particle used in any of the methods described herein can also include a sequence that encodes a selectable marker and/or a sequence that encodes an antibiotic resistance gene. Selectable marker genes are known in the art and include, without limitation, galactokinase, beta-galactosidase, chloramphenicol acetyltransferase, beta lactamase, green fluorescent protein (GFP), alkaline phosphate, etc. Antibiotic resistance genes are also known in the art and include, without limitation, ampicillin, streptomycin, spectromycin, etc. A number of suitable empty amplicon vectors have previously been described in the art including, without limitation, pHSVIac (ATCC Accession 40544; U.S. Pat. No. 5,501,979; Stavropoulos and Strathdee, J. Virol., 72:7137-43, 1998), and pHENK (U.S. Pat. No. 6,040,172). The pHSVIac vector includes the HSV-1 a segment, the HSV-1c region, an ampicillin resistance marker, and an E. coli lacZ marker. The pHENK vector includes the HSV-1 a segment, an HSV-1 ori segment, an ampicillin resistance marker, and an E. coli LacZ marker under control of the promoter region isolated from the rat preproenkephalin gene (i.e., a promoter operable in brain cells). The sequences encoding a selectable marker, the sequences encoding the antibiotic resistance gene (which may also serve as a selectable marker), and the sequences encoding the transgene, may be under the control of regulatory sequences such as promoter elements that direct the initiation of transcription by RNA polymerase, enhancer elements, and suitable transcription terminators or polyadenylation signals. Preferably, the promoter elements are operable in the cells of the patient that are targeted for transformation. A number of promoters have been identified that are capable of regulating expression within a broad range of cell types. These include, without limitation, HSV immediate-early 4/5 (IE4/5) promoter, cytomegalovirus (“CMV”) promoter, SV40 promoter, and P-actin promoter. Likewise, a number of other promoters have been identified that can regulate expression within a narrow range of cell types. These include, without limitation, the neural-specific enolase (NSE) promoter, the tyrosine hydroxylase (TH) promoter, the GFAP promoter, the preproenkephalin (PPE) promoter, the myosin heavy chain (MHQ promoter), the insulin promoter, the cholineacetyltransferase (ChAT) promoter, the dopamine β-hydroxylase (DBH) promoter, the calmodulin dependent kinase (CamK) promoter, the c-fos promoter, the c-jun promoter, the vascular endothelial growth factor (VEGF) promoter, the erythropoietin (EPO) promoter, and the EGR-I promoter. The transcription termination signal should, likewise, be operable in the cells of the patient that are targeted for transformation. Suitable transcription termination signals include, without limitation, polyA signals of HSV genes such as the vhs polyadenylation signal, SV40 poly-A signal, and CW IE1 polyA signal.

Conditions Amenable to Treatment

The compositions of the present invention (including herpesvirus particles and cells that contain them) can be used to treat: (1) patients who have been, or who may become, infected with a wide variety of agents (including viruses such as a human immunodeficiency virus, human papilloma virus, herpes simplex virus, influenza virus, pox viruses, bacteria (including eubacteria and archaea), such as E. coli or a Staphylococcus, a parasite, a mycoplasma, or an unconventional infectious agent such as a prion protein), (2) patients with a wide variety of cancers; (3) patients with a neurological disease or disorder; and (4) patients who have or who may experience hearing loss (a patient “with” a disorder can be a patient diagnosed as having that disorder). A patient can be treated after they have been diagnosed as having a cancer, an infectious disease, or a neurological disorder or, since the agents of the present invention can be formulated as vaccines, patients can be treated before they have developed the cancer, infectious disease or neurological disorder. Thus, “treatment” encompasses prophylactic treatment. Similarly, patients who have experienced a loss of hearing can be treated at any time, including before the loss occurs (e.g., hf-HSV amplicon particles can be administered before the patient is exposed to some agent, such as a chemotherapeutic agent or industrial hazard, that may damage one or more of their senses, including their hearing).

With respect to cancer in general and leukemia in particular, we note that chronic lymphocytic leukemia (CLL) is a malignancy of mature appearing small B lymphocytes that closely resemble those in the mantle zone of secondary lymphoid follicles (Caligaris-Cappio and Hamblin, J. Clin. Oncol. 17:399-408, 1999). CLL remains a largely incurable disease of the elderly with an incidence of more than 20 per 100,000 above the age of 70, making it the most common leukemia in the United States and Western Europe. CLL, which arises from an antigen-presenting B cell that has undergone a non-random genetic event (del13q14-23.1, trisomy 12, del11 q22-23 and del6q21-23 (Dohner et al., J. Mol. Med. 77:266-281, 1999)) and clonal expansion, exhibits a unique tumor-specific antigen in the form of surface immunoglobulin. CLL cells possess the ability to successfully process and present this tumor antigen, a characteristic that makes the disease an attractive target for immunotherapy (Bogen et al., Eur. J. Immunol. 16:1373-1378, 1986; Bogen et al., Int. Rev. Immunol. 10:337-355, 1993; Kwak et al., N. Engl. J. Med. 327:1209-1215, 1992; and Trojan et al., Nat. Med. 6:667-672, 2000). However, the lack of expression of co-stimulatory molecules on CLL cells renders them inefficient effectors of T cell activation, a prerequisite for generation of anti-tumor immune responses (Hirano et al., Leukemia 10: 1168-1176, 1996). This failure to activate T cells has been implicated in the establishment of tumor-specific tolerance (Cardoso et al., Blood 88:41-48, 1996). Reversal of preexisting tolerance can, potentially, be achieved by up-regulating a panel of co-stimulatory molecules (B7.1, B7.2 and ICAM-I) (Grewal and Flavell, Immunol. Rev. 153:85-106, 1996) through the activation of CD40 receptor-mediated signaling and concomitant enhancement of antigen presentation machinery (Khanna et al., J. Immunol. 159:5982-5785, 1997; Lanzavecchia, Nature 393:413414, 1998; Diehl et al., Nat. Med. 5:774-779, 1999; Sotomayor et al., Nat. Med. 5:780-787, 1999).

Applying the information above in effective gene therapies for CLL has been hampered by the lack of a safe and reliable vector that can be used to transduce primary leukemia cells. In contrast to tumor cell lines, CLL cells are effectively post-mitotic; only a small fraction of the population enters the cell cycle (Andreeff et al, Blood 55:282-293, 1980). Although both retroviral and adenoviral vectors have been employed in different clinical trials for cancer gene therapy, both systems exhibit limitations (Uckert and Walther, Pharmacol. Ther. 63:323-347, 1994; Vile et al., Mol. Biotechnol. 5:139-158, 1996; Collins, Ernst Schering Research Foundation Workshop, 2000; Hitt et al., Adv. Pharmacol. 40:137-206, 1997; Kochanek, Hum. Gene Ther. 10:2451-2459, 1999). For example, the low levels of integrin receptors for adenovirus on CLL cells mandates the use of very high adenovirus titers, preactivation of the CLL cell with IL-4 and/or anti-CD40/CD40L (Cantwell et al., Blood 88:4676-4683, 1996; Huang et al., Gene Ther. 4:1093-1099, 1997), or adenovirus modification with polycations to achieve clinically meaningful levels of transgene expression (Howard et al., Leukemia 13:1608-1616, 1999).

In some of the Examples below, HSV amplicon particles were used to transduce primary human B-cell chronic lymphocytic leukemia (CLL) cells. The vectors were constructed to encode β-galactosidase (by inclusion of the lacZ gene), B7.1 (also known as CD80), or CD40L (also known as CD154), and they were packaged using either a standard helper virus (HSVlac, HSVB7.1, and HSVCD40L) or by a helper virus-free method (hf-HSVlac, hf-HSVB7.1, and hf-HSVCD40L). CLL cells transduced with these vectors were studied for their ability to stimulate allogeneic T cell proliferation in a mixed lymphocyte tumor reaction (MLTR). A vigorous T cell proliferative response was obtained using cells transduced with hf-HSVB7.1 but not with HSVB7.1. CLL cells transduced with either HSVCD40L or hf-HSVCD40L were also compared for their ability to up-regulate resident B7.1 and function as T cell stimulators. Significantly enhanced B7.1 expression was seen in response to CD40L delivered by hf-HSVCD40L amplicon stock (compared to HSVCD40L). CLL cells transduced with hf-HSVCD40L were also more effective at stimulating T cell proliferation than those transduced with HSVCD40L stocks. These studies support the conclusion that HSV amplicons are efficient vectors for gene therapy, particularly of hematologic malignancies, and that helper virus-free amplicon preparations are better suited for use in therapeutic compositions.

Neuronal diseases or disorders that can be treated include lysosomal storage diseases (treatment can occur, for example, by expressing MPS I-VIII, hexoaminidase A/B, etc.), Lesch Nyhan syndrome (treatment can occur, for example, by expressing HPRT), amyloid polyneuropathy (treatment can occur, for example, by expressing B-amyloid converting enzyme (BACE) or amyloid antisense sequences), Alzheimer's Disease (treatment can occur, for example, by expressing a nerve growth factor such as NGF, ChAT, BACE, etc.), retinoblastoma (treatment can occur by, for example, expressing pRB), Duchenne's muscular dystrophy (treatment can occur by expressing Dystrophin), Parkinson's Disease (treatment can occur, for example, by expressing GDNF, BcI-2, TH, AADC, VMAT, sequences antisense to mutant alpha-synuclein, etc.), Diffuse Lewy Body disease (treatment can occur, for example, by expressing a heat shock protein, parkin, or antisense or RNAi molecules to alpha-synuclein), stroke (treatment can occur by, for example, expressing Bcl-2, HIF-DN, BMP7, GDNF, or other growth factors), brain tumor (treatment can occur by, for example, expressing angiostatin, antisense VEGF, antisense or ribozyme to EGF or scatter factor, or pro-apoptotic proteins), epilepsy (treatment can occur by, for example, expressing GAD65, GAD67, or pro 10 apoptotic proteins into focus), or arteriovascular malformation (treatment can occur by expressing proapoptotic proteins).

Therapeutic Agents

As noted above, the herpesvirus amplicon particles described herein (and the cells that contain them) can express a heterologous protein (i.e., a full-length protein or a portion thereof (e.g., a functional domain or antigenic peptide) that is not naturally encoded by a herpesvirus). The heterologous protein can be any protein that conveys a therapeutic benefit on the cells in which it, by way of infection with a herpesvirus amplicon particle, is expressed or a patient who is treated with those cells.

The therapeutic agents can be immunomodulatory (e.g. immunostimulatory) proteins (as described in U.S. Pat. No. 6,051,428). For example, the heterologous protein can be an interleukin (e.g., IL-1, IL-2, IL-4, IL-10, or IL-15), an interferon (e.g., IFNγ), a granulocyte macrophage colony stimulating factor (GM-CSF), a tumor necrosis factor (e.g., TNFα), a chemokine (e.g., RANTES, MCP-1, MCP-2, MCP-3, DC-CK1, MIP-1α, MIP-3α, MIP-β, MIP-3β, an α or C-X-C chemokine (e.g., IL-8, SDF-1β, 8DF-1α, GRO, PF-4 and MIP-2). Other chemokines that can be usefully expressed are in the C family of chemokines (e.g., lymphotactin and CX3C family chemokines).

Intercellular adhesion molecules are transmembrane proteins within the immunoglobulin superfamily that act as mediators of adhesion of leukocytes to vascular endothelium and to one another. The vectors described herein can be made to express ICAM-1 (also known as CD54), and/or another cell adhesion molecule that binds to T or B cells (e.g., ICAM-2 and ICAM-3).

Costimulatory factors that can be expressed by the vectors described herein are cell surface molecules, other than an antigen receptor and its ligand, that are required for an efficient lymphocytic response to an antigen (e.g., B7 (also known as CD80) and CD40L).

When used for gene therapy, the transgene encodes a therapeutic transgene product, which can be either a protein or an RNA molecule.

Therapeutic RNA molecules include, without limitation, antisense RNA, inhibitory RNA (RNAi), and an RNA ribozyme. The RNA ribozyme can be either cis or trans acting, either modifying the RNA transcript of the transgene to afford a functional RNA molecule or modifying another nucleic acid molecule. Exemplary RNA molecules include, without limitation, antisense RNA, ribozymes, or RNAi to nucleic acids for huntingtin, alpha synuclein, scatter factor, amyloid precursor protein, p53, VEGF, and others.

Therapeutic proteins include, without limitation, receptors, signaling molecules, transcription factors, growth factors, apoptosis inhibitors, apoptosis promoters, DNA replication factors, enzymes, structural proteins, neural proteins, and histone or non-histone proteins. Exemplary protein receptors include, without limitation, all steroid/thyroid family members, nerve growth factor (NGF), brain derived neurotrophic factor (BDNF), neutotrophins 3 and 4/5, glial derived neurotrophic factor (GDNF), cilary neurotrophic factor (CNTF), persephin, artemin, neurturin, bone morphogenetic factors (BMl's), c-ret, gp 130, dopamine receptors (D ID5), muscarinic and nicotinic cholinergic receptors, epidermal growth factor (EGF), insulin and insulin-like growth factors, leptin, resistin, and orexin. Exemplary protein signaling molecules include, without limitation, all of the above-listed receptors plus MAPKs, ras, rac, ERKs, NFKβ, GSK3β, AKT, and PI3K. Exemplary protein transcription factors include, without limitation, ˜300, CBP, HIF-lalpha, NPAS1 and 2, HIF-1β, p53, p73, nurr 1, nurr 77, MASHs, REST, and NCORs. Exemplary neural proteins include, without limitation, neurofilaments, GAP43, SCG-10, and others. Exemplary enzymes include, without limitation, TH, DBH, aromatic amino acid decarboxylase, parkin, unbiquitin E3 ligases, ubiquitin conjugating enzymes, cholineacetyltransferase, neuropeptide processing enzymes, dopamine, VMAT and other catecholamine transporters. Exemplary histones include, without limitation, Hl-5. Exemplary non-histones include, without limitation, ND10 proteins, PML, and HMG proteins. Exemplary pro- and anti-apoptotic proteins include, without limitation, bax, bid, bak, bcl-xs, bcl-xl, bcl-2, caspases, SMACs, and IAPs.

Formulation and Administration of Herpesvirus Amplicon Particles

The herpesvirus amplicon particles described herein can be administered to patients directly or indirectly; alone or in combination with other therapeutic agents; and by any route of administration. For example, the herpesvirus HSV amplicon particles can be administered to a patient indirectly by administering cells transduced with the vector to the patient. Alternatively, or in addition, a herpesvirus amplicon particle could be administered directly. For example, a herpesvirus amplicon particle that expresses an immunostimulatory protein or a tumor-specific antigen can be introduced into a tumor by, for example, injecting the vector into the tumor or into the vicinity of the tumor (or, in the event the cancer is a blood-borne tumor, into the bloodstream).

Administration of HSV-immunomodulatory protein amplicons encoding cytokines such as IL-2, GM-CSF and RANTES, intercellular adhesion molecules such as ICAM-1 and costimulatory factors such as B7.1 all provide therapeutic benefit in the form of reduction of preexisting tumor size, a vaccine-effect protecting against tumor growth after a subsequent challenge, or both (see U.S. Pat. No. 6,051,428; see also Kutubuddin et al., Blood 93:643-654, 1999). The helper virus-free HSV vectors disclosed herein can be administered in the same manner.

The herpesvirus amplicon particles described herein, and cells that contain them, can be administered, directly or indirectly, with other species of HSV-transduced cells (e.g., HSV-immunomodulatory transduced cells) or in combination with other therapies, such as cytokine therapy. Such administrations may be concurrent or they may be done sequentially. Thus, in one embodiment, HSV amplicon particles, the vectors with which they are made (i.e., packaging vectors, amplicon plasmids, and vectors that express an accessory protein) can be injected into a living organism or patient (e.g., a human patient) to treat, for example, cancer or an infectious disease. In further embodiments, one or more of these entities can be administered after administration of a therapeutically effective amount of a cytokine.

The concentrated stock of HSV amplicon particles is effectively a composition of the HSV amplicon particles in a suitable carrier. HSV amplicon particles can also be administered in injectable dosages by dissolving, suspending, or emulsifying them in physiologically acceptable diluents with a pharmaceutical carrier (at, for example, about 1×10⁷ amplicon particles per ml). Titers can be higher, however. For example, titers can be 1×10⁸ to 5×10⁸, or even higher (e.g., 1×10⁹ to 5×10⁹). Such carriers include sterile liquids, such as water and oils, with or without the addition of a surfactant and other pharmaceutically and physiologically acceptable carriers, including adjuvants, excipients or stabilizers. The oils that can be used include those obtained from animals or vegetables, petroleum based oils, and synthetic oils. For example, the oil can be a peanut, soybean, or mineral oil. In general, water, saline, aqueous dextrose and related sugar solutions, glycols (e.g., propylene glycol or polyethylene glycol) are preferred liquid carriers, particular when the amplicon particles are formulated for administration by injection.

For use as aerosols, the HSV amplicon particles, in solution or suspension, can be packaged in a pressurized aerosol container together with suitable propellants, for example, hydrocarbon propellants like propane, butane, or isobutene with conventional adjuvants. The particles can also be administered in a non-pressurized form such as in a nebulizer or atomizer.

Use of the amplicon as a diagnostic tool. The amplicon particles of the invention can also be employed as diagnostic tools in a wide variety of situations (i.e., in the diagnosis of cancer or infectious disease). For example, in the case of HIV, white blood cells (WBCs) could be isolated from a patient's blood and transduced with an HSV amplicon vector containing a tat-responsive element (TAR) and a reporter gene (i.e., lacZ or placental alkaline phosphatase). If the WBCs harbor HIV virions, the strong HIV transactivator protein ‘tat’ will be present and will bind to the TAR element engineered into the HSV amplicon. This, in turn, will lead to activation of reporter gene expression that can be detected by a standard colorimetric assay. This diagnostic method could be employed for detecting any infectious virus that expresses a strong transcriptional activator protein and where the cognate cis responsive elements for this activator protein are already known. Examples of other viruses for which this method could be employed are Epstein Barr virus (EBV) and any immunodeficiency virus (e.g., simian immunodeficiency virus (SIV) or feline immunodeficiency virus (FIV)).

### EXAMPLES

The studies that follow were designed to test the ability of HSV amplicon particles, generated using helper viruses bearing mutations in VP16 and VHS, to transfer genes into neurons of the CNS. While neither mutation prevented packaging, amplicon titers were reduced 16 and 18-fold for VP16 (14HD3) and VHS (D3vhsZ) generated stocks. The addition of wild-type VP16 in trans during packaging improved 14HD3 amplicon titers 20-fold in a TAATGARAT-independent manner and occurred in part through a mechanism that increased the fraction of amplicon units capable of supporting transcription. The mRNAse function of VHS was dispensable for the observed trans-induction of vhs mutant virus packaged amplicon vectors. Most strikingly, 14HD3vhsZ-packaged amplicon stocks, enhanced with both wild-type VP16 and mutant VHS, demonstrated markedly less toxicity on cortical neuronal cultures when compared to controls harboring wild-type VHS. These studies demonstrate that restricted replication combined with the use of mRNAse deficient vhs mutants can significantly improve the toxicity profiles of helper-containing amplicon vector stocks.

The viral proteins VP16 and VHS, which are delivered at the time of infection, help regulate the temporal sequence of viral replication by stimulating de novo expression from the viral genome and co-opting the host cell's biosynthetic machinery (Dargan and Subak-Sharpe, Virol. 239:378-388, 1997). Because we thought these proteins might contribute to vector toxicity in neurons (e.g., cortical neurons), we investigated whether helper viruses mutated in either VP16 or VHS function could complement amplicon particle packaging and reduce the toxicity profiles of packaged stocks when used at high multiplicities of infection in a neuronal culture system. Using multi-mutated helper viruses, we have demonstrated that the simultaneous manipulation of both VP16 and VHS activity generates vector titers comparable to single deletion systems while substantially limiting vector toxicity.

The ability to generate HSV amplicon virus requires that the amplicon plasmid, which contains the origin of replication (ori) and packaging (“a”) sequences, compete with the full-length helper virus genome for the machinery involved in generating infectious viral particles. However, due to inefficiencies in delivering the amplicon DNA to the packaging cell line, a sizeable proportion of packaging cells, once super-infected, generate only progeny helper virus, resulting in lower amplicon to helper ratios. To address this problem multiple-mutant helper viruses were incorporated in the packaging process and virus production was restricted to those cells receiving both the amplicon plasmid and plasmid(s) capable of complementing the additional mutation(s) encoded by the helper virus. This strategy effectively boosted the amplicon to helper ratios for stocks generated using both VP16 and VHS deleted helper viruses approximately 1.5 to 3-fold, while effectively raising the amplicon concentration to levels obtained with the IE3-deleted parental vector D30EBA. Interestingly, it was noted, using the helper-free system, that addition of expression plasmids for VP16 and VHS at the time of packaging enhances the titers generated 50-fold and reduced the pseudo-transduction phenomenon commonly associated with first-generation helper-free systems (Bowers et al., Gene Ther. 8:111-120, 2001).

The tegument protein VP16 participates in the regulation of de novo viral gene expression, packaging of the viral genome into the nucleocapsid, and is required for the generation of infectious progeny virus. Addition of the VP16 protein in trans at the time of vector packaging produced a greater enrichment in total amplicon particles compared to VHS-augmented stocks. While this effect did not appear to require the presence of the VP16 binding elements (TATGARAT) (SEQ ID NO:3) within the amplicon plasmid, the data suggest that VP16 promotes the transcriptional competence of the amplicon template once delivered, effectively “unmasking” a latent population of infectious virus. The fact that the de-acetylase inhibitor TSA was ineffective in enhancing the titers of amplicon stocks containing wild-type VP16 suggests that VP16 may alter the post-translational modification of either pre-assembled or newly assembled nucleo-protein complexes associated with the episomal amplicon transcription unit. In support of this concept, VP16 has been shown in vitro to inhibit the activity of histone de-acetylase activity (HDAC) on templates supporting GAL4 transcription (Utley et al., Nature 394:498-502, 1998). Alternatively, the presence of wildtype VP16 within the packaged amplicon may influence the sub-cellular trafficking of the nucleocapsid-genome unit. This may occur either actively by directing the nucleocapsid-genome unit to the nuclear import machinery, or passively through the inhibition of degradation pathways. Additional modifications occurring at the time of vector packaging may also result in the functional silencing of the amplicon transcription unit. The examples that follow provide a better understanding of the interactions between the viral transgene and host-factors and of the mechanism by which VP16 resists cell-mediated vector silencing. As such, they help to explain why it has been so difficult to achieve sustained viral transgene expression in vivo.

Although optimizing amplicon yield and amplicon to helper ratios were goals in this study, the primary objective was to identify a packaging protocol capable of producing reasonably high titer vector with attenuated vector-mediated cytotoxicity. Theoretical sources of toxicity within HSV-1 amplicon vector stocks include toxic proteins either delivered at the time of infection or expressed de novo from the helper genome, and toxic metabolites co-purified at the time of virus collection. Such contaminants include excitotoxins, which potently induce necrotic cell death in exposed neurons within hours of exposure (Ankarcrona et al., Neuron 15:961-973, 1995), and can be inhibited with the use of the glutamate antagonist kynurenic acid (Ho et al., J. Neurosci. Meth. 57:205-215, 1995). In the current experiments, excitotoxicity did not clearly distinguish vector stocks from each other. The cell death profiles of primary cultures treated with UV-inactivated virus (a treatment that limits gene expression from the viral genome) were equivalent amongst sister cultures infected with single (D30EBA), double (14HΔ3, Δ3vhsZ) and triple (14HΔ4vhsZ) deleted helper viruses. Furthermore, the toxicity induced by non-irradiated amplicon stocks continued to progress beyond the first day post-infection, consistent with the late effects of de novo gene expression from the viral genome and not of an acute necrotic process (Johnson et al., J. Virol. 68:6347-6362, 1994).

Several strategies have been devised to limit the toxicity arising from de novo gene expression from the viral genome. Recombinant HSV-1 vectors in which therapeutic constructs are delivered within the context of the herpes genome, deleted of VP16 and viral immediate-early genes are capable of generating high-titer stocks with attenuated toxicity profiles (Krisky et al., Gene Ther. 5:1593-1603, 1998; unlike the present invention, where helper viruses are used to deliver amplicon particles, others have used helper viruses to deliver proteins). Although not directly measured, we believe the reduction in toxicity achieved with the hybrid system of the invention stems in part from the VP16ⁱⁿ mutation. This approach, however, exposes the transduced cell to the residual expression of other potentially damaging gene products from intact portions of the HSV genome. Although recombinant HSV-1 vectors have not been tested in the system described herein, it is expected that amplicon vectors packaged by restricted replication would deliver a fraction of these toxic proteins given the high amplicon to helper ratios obtained. Alternatively, cosmid and bacterial artificial chromosome (BAC) DNA-based systems have been developed for use in the generation of helper-free amplicon plasmids. However, these strategies are hampered by their inability to produce viral titers comparable to those obtained with either recombinant or helper-based amplicon systems (Fraefel et al., J. Virol. 70:7190-7197, 1996).

Compared to viral stocks containing the VP16 insertion mutant and wild-type vhs, amplicon vectors packaged using helper virus containing mutant forms of both vp16 and vhs exhibit reduced toxicity on neuronal cultures. Because the VHS protein encodes both catalytic and structural functions and regulates the transcriptional activity of VP16, the influence of VHS on viral titers and toxicity likely represents a summation of these activities, making such studies difficult to interpret. Therefore mRNAse-defective point mutants (M384 and SC243) were utilized that retained the ability to both limit the transcriptional activity of VP16 and to be packaged into the viral tegument (Jones et al., J. Virol. 69:4863-4871, 1995). We found that delivery of the vhs mutants in trans at the time of vector packaging provided a significant survival advantage to transduced cultures when compared to control stocks.

VHS has also been identified as a virulence factor in the course of natural infection (Strelow and Leib, J. Virol. 69:6779-6786, 1995). Deletion of vhs from the genome disrupts the efficient generation of viral titers and enhances the ability of the infected cell population to mount protective cell-autonomous and cell-mediated responses to microbial infection (Suzutani et al., J. Gen. Virol. 81:1763-1771, 2000). VHS mutations that lead to abolished RNAse activity are the R27, Sc243, and M384 mutations have been described previously by Jones et al. (J. Virol. 69:4863-4871, 1995). The mutated forms of VHS can be incorporated into the tegument of the viral particle to serve a structural role but are unable to carry out the RNAse function.

In summary, these findings outline a novel approach for producing HSV-1 viral vectors with reduced in vitro toxicity, and characterize the underlying mechanism for the observed differences in stock titer and toxicity profiles for HSV-1 vectors generated using the restricted replication method. Through the addition of wild-type VP16 and mutant VHS HSV-1 proteins in trans to an amplicon packaging protocol utilizing helper viruses harboring genomic mutations in both VP16 and VHS, improvements were demonstrated in amplicon titers, the amplicon to helper virus (A:H) titer ratios, and markedly reduced toxicity on primary cortical neuronal cultures. These data support a neurotoxic role for VHS in the setting of amplicon infection and demonstrate that replacement of mutant for wild-type VHS improves the toxicity profile of not only the HSV-1 amplicon, but also for other herpes-based gene delivery systems.

The following reagents and methods were used to conduct the studies described below.

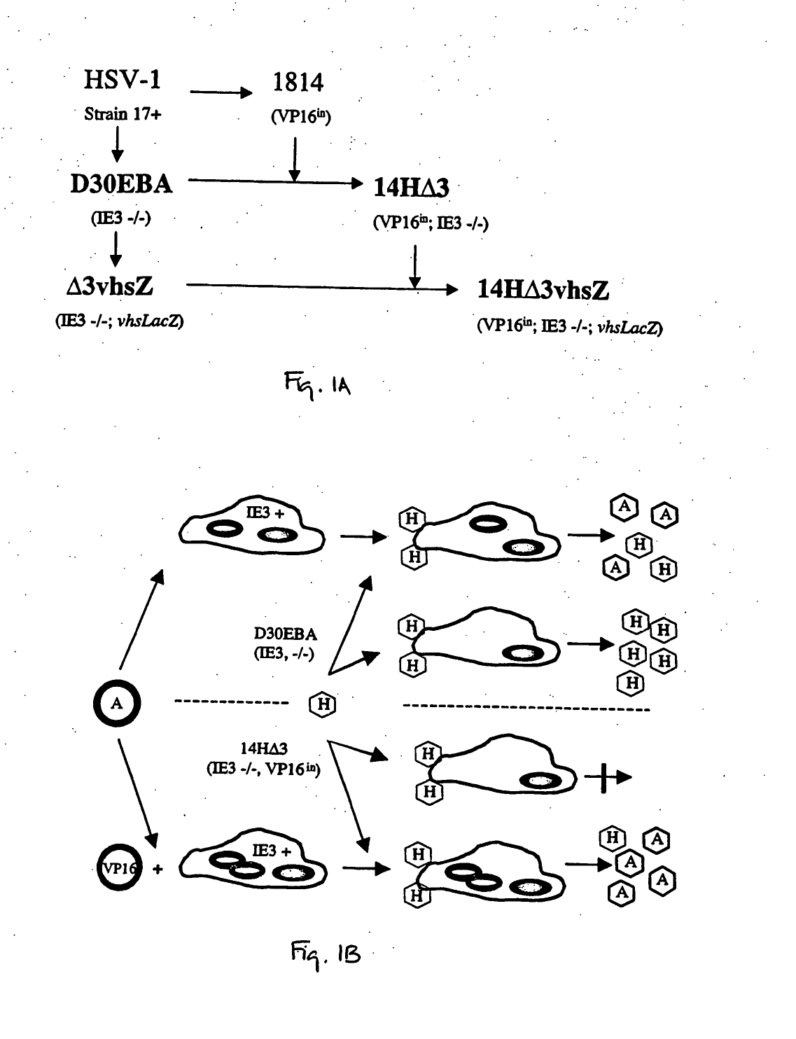

Cell lines and virus stocks. The RR1 packaging cell line stably expressing the HSV IE3 gene was maintained in DMEM/10% FCS with G418 (200 mg/ml) (Life Technologies, Gaithersburg, Md.). Vero and NIH 3T3 cell lines, obtained from the American Type Culture Collection (ATCC; Manassas, Va.), were maintained in DMEM with 5% and 10% FCS, respectively. The construction of the helper viruses Δ3vhsZ, 14HΔ3 and 14HΔ3vhsZ (FIG. 1A) kindly provided by P. Johnson (Neurovir Inc., San Diego, Calif.) are described in detail elsewhere (Paterson and Everett, J. Gen. Virol. 71:1775-1783, 1990; Johnson et al., J. Virol. 68:6347-6362, 1994; these publications are hereby incorporated by reference in the present application in their entirety). Briefly, the Δ3vhsZ virus has been deleted of IE3 expression and contains a cytomegalovirus (CMV)/β-galactosidase (lacZ) expression cassette inserted at the Nru I site of VHS, resulting in the elimination of residues 237-489 which encompass the VP16 binding domain (Jones et al., J. Virol. 69:4863-4871, 1995; hereby incorporated by reference in its entirety). The 14HΔ3 virus was generated by crossing the HSV-1 linker insertion mutant VP16¹⁸¹⁴ into the IE3-deficient D30EBA helper background (Ace et al., J. Virol. 63:2260-2269, 1989). This mutation further attenuates IE gene transactivation by disrupting the ability of VP16 to associate with cellular transcription associated factors (OCT1, HCF) or to bind DNA. The VP16¹⁸¹⁴ mutation can be complemented by the addition of 1 mM HMBA (SIGMA-Aldrich, St. Louis, Mo.). The triple mutant 14HΔ3vhsZ was made by crossing the VP16ⁱⁿ and Δ3vhsZ viruses (FIG. 1A).

Virus preparation, titering and complementation assays. Prior to use in amplicon packaging, helper virus was plaque purified twice on RR1 cells and large-scale stocks were prepared in the presence of 1 mM HMBA. All helper stocks were titered and screened for recombination in the presence of HMBA on complementing (RR1) and non-complementing cell lines (Vero). To study the influence of VP16 and/or VHS on amplicon production, a small-scale packaging format performed in six-well plates allowed for replicate generation of viral stocks (n=3). Small-scale vector packaging was performed using 1×10⁶ RR1 cells plated to 28 cm² wells (6-well plates) followed by transfection of amplicon, VP16 and/or VHS-encoding DNAs (0.5 μg each) with the carrier plasmid pRcCMV (Invitrogen, Hercules, Calif.) as indicated by Lipofectamine-mediated transfection (Lifetechnologies, Gaithersburg, Md.). Twenty-four hours later, cultures were super-infected with helper virus at an MOI of 0.1. Forty-eight hours after super-infection, cytopathic monolayers were harvested, freeze-thawed, sonicated and viral supernatants were clarified of cellular debris by centrifugation (1,200 rpm/10 min/4° C.) prior to storage at −80° C. without concentration. Green fluorescent units (GFU/ml) determined for viral stocks made using the dual ORF expression plasmid HSV4/5lucCMVegfp. Helper titers were determined on the RR1 packaging in the presence of 1 mM HMBA.

Vectors used in cortical neuronal cytotoxicity studies were prepared as follows: RR1 packaging cells (3×10⁶ cells/60 mm dish) were transfected with reporter, VP16 and/or VHS expression constructs using pRcCMV to balance to a total of 3 μg DNA (1.0 μg each), super-infected 24 hours later at an MOI of 0.1, and harvested 48 hours post infection. Monolayers were harvested, sonicated, clarified and purified without re-passage by ultra-centrifugation over a 25% sucrose cushion on a Sorvall RC5 ultracentifuge (24,000 rpm/2 hr/4° C.). Viral pellets were suspended in PBS (Mg²⁺/Ca²⁺-containing) and stored at −80° C.

Amplicon titers were determined by transducing 1×10⁵ NIH3T3 cells plated on 12 mm cover slips with a viral dilution series, and fixed 24 hours later with paraformaldehyde (4%, PBS pH 7.6). Cover slips were inverted in mowiol and GFP titers were scored by standard FITC fluorescence. Titers for vectors containing the plasmids HSVlac and CMVminOrislac, which express β-galactosidase, were titered by X-gal staining (5 mM potassium ferricyanide; 5 mM potassium ferrocyanide; 0.02% NP-40; 0.01% sodium deoxycholic acid; 2 mM MgCl₂ and 1 mg/ml X-gal dissolved in PBS).

Plasmid constructs. The plasmids pucCMVvhs^{wt}, pCMVvhsM384 pucCMVvhsSC243, and pEVRF (containing a CMV-driven VP16 ORF from strain 17⁺) were kindly provided by Dr. J. Smiley (University of Alberta, Edmonton, Alberta). The vector pBSIIvhs was generated by subcloning a 3.5-kb Hpa I/Hind III fragment from the cos56 cosmid vector encompassing the UL41 open reading frame and its native 5′ and 3′ transcriptional regulatory elements. The amplicon reporter plasmid HSVlac contains the bacterial lacZ gene under the control of either the HSV immediate-early (IE) 4/5 promoter, which contains native elements from the HSV-1 KOS promoter region of the IE4 and IE5 genes (Geller et al., Proc. Natl. Acad. Sci. USA 87:8950-8954, 1990). The CMVminOrisLac amplicon contains a minimal 237 bp HSV origin of replication deleted of Oct-1 and TAATGARAT (SEQ ID NO:2) sequences, and a 600-bp segment of the human CMV promoter upstream of the bacterial lacZ gene (Lu and Federoff, Human Gene Therapy 6:419-428, 1995).

The vector HSVlucCMVegfp, which expresses both luciferase and the enhanced-green fluorescent protein under control of the HSV IE 4/5 and human CMV promoters, respectively, was constructed as follows. The CMVegfp transcription unit flanked by Acc I sites was generated by PCR using the plasmid pC2EGFP (Clontech, Palo Alto, Calif.). In addition to encoding the necessary restriction sites, 5′ and 3′ termini were engineered to include Kozak consensus start sequence and termination sequences, respectively, and the fragment was ligated into the Acc I site of HSVprPuc. The open reading frame for luciferase was transferred from the vector pGL3control (Promega, Madison, Wis.) as a Hind III/Xba I fragment into the corresponding sites of HSVprPucCMVegfp.

Quantitative genome analysis. Twelve hours after plating (1×10⁶ cells/6 wells), Vero cells were infected in triplicate with 200 μl of amplicon virus for 12 hours, rinsed twice with D-PBS and growth media was replaced. Forty-eight hours post-infection, DNA was harvested by phenol:chloroform extraction and real-time quantitative PCR was performed on 400 ng of genomic DNA using “TaqMan™” chemistry and the PE Model 7700 Sequence Detection System (PE Applied Biosystems, Foster City, Calif.) (Brewer, J. Neurosci. Res. 42:674-683, 1995). Reactions were prepared as follows: 1X Universal Master Mix, 900 nM forward primer, 900 nM reverse primer, and 10 nM probe for 50° C. for 5 minutes, 95° C. for 5 minutes, and 40 cycles at 95° C. for 15 seconds then 60° C. for 1 minute. Primer and probe sequences were as follows LacZ: Fwd: 5′-30 GGGATCTGCCATTGTCAGACAT-3′ (SEQ ID NO:4), Rev: 5′-TGGTGTGGGCCATAATTCAA-3′ (SEQ ID NO:5), Probe: 5′-FAM-ACCCCGTACGTCTTCCCGAGCG-TAMRA-3′ (SEQ ID NO:6). TaqMan probes against the β-actin coding sequence were used in the same reaction to normalize against total genomic DNA Fwd: 5′-GCCTTTTTGCAGCT CCTTCGT-3′ (SEQ ID NO:7), Rev: 5′-CCAGCGCAGCGATATCG-3′ (SEQ ID NO:8), Probe: 5′-VIC-CGCCACCAG TTCGCCATGGA-TAMRA-3′ (SEQ ID NO:9). Standard curves for lacZ and genomic DNA were generated using HSVlac plasmid DNA or genomic DNA from non-transduced cells.

TSA studies. The histone de-acetylase inhibitor trichostatin A (TSA; Sigma Chemical Co., St. Louis, Mo.) was added to NIH 3T3 monolayers to a final concentration of 0.5 μg/ml at the time of infection and VP16−/+ viruses expressed for 24 hours prior to titering.

Toxicity Studies. Primary cortical neurons were harvested from E15 mice and were prepared according to published methods (Brewer, J. Neurosci. Res. 42:674-683, 1995). Cortices were dissociated by trypsinization (0.25% trypsin/EDTA) for 15 minutes at 37° C. and washed twice with HBSS containing Ca²⁺ and Mg²⁺. Cells were dissociated further by mechanical dissociation with a serologic pipette and resuspended in serum-free Neurobasal® plating medium containing 0.5 mM L-glutamine, 3.7 μg/ml L-glutamic acid, and 2% v/v B-27 supplement (Life Technologies, Gaithersburg, Md.). Ninety-six well plates were coated with poly-D-lysine (15 μg/ml; Sigma), rinsed twice with sterile water, and cells were plated at a density of 4×10⁴ cells per well. Cultures were maintained at 37° C. in a 6% CO₂ environment, infected and analyzed for viability using the MTS assay (Promega, Madison, Wis.). To inactivate helper gene expression, viral aliquots were treated in a Stratalinker (model 1800) with 160 mJoules/cm² of 254 nm of UV light, which interferes with both replication and transcription of the viral DNA but has no affect on the infectivity of the virus (Johnson et al., J. Virol. 66:2952-2965, 1992). The UV-mediated reduction in helper titers was conformed by plaque assay performed in the presence of 1 mM HMBA.

Data and statistical analysis. Small-scale vector packaging experiments were performed in triplicate and the amplicon to helper rations (A:H) were determined by pairing amplicon and helper titers derived from identical samples. In all cases, data are presented as the average±standard deviation for both experiments. Statistical testing was performed using the two-tailed students t-test assuming unequal variances.

### Example 1

### Addition of VP16 in Trans Improves Amplicon Titers

To improve amplicon to helper (A:H) titer ratios and limit toxicity imparted by the helper virus genome, a packaging method was developed that can be carried out as a single-round transfection-infection procedure. The method utilizes a series of HSV-1 viruses deficient in wild-type VP16 and/or VHS function (FIG. 1A). By providing the IE3 gene in trans, the RR1 stable cell line effectively complements replication of IE3-deleted D30EBA helper virus and packaging of amplicon-containing virions in cells containing the transfected amplicon DNA (FIG. 1B, top). The Δ3vhsZ, 14HΔ3 and 14HΔ3vhsZ viruses contain additional mutations introduced into the D30EBA background limiting their ability to support virus production in the RR1 line. However, by co-transfecting expression constructs that complement the additional helper mutations (VP16ⁱⁿ as in the case of 14HΔ3; VHS as in the case of Δ3vhsZ) along with the amplicon vector, virus propagation is biased toward the packaging of amplicon DNA, while the propagation of helper virus in non-transfected cells is prohibited (FIG. 1B, bottom). While VP16 and/or VHS can be expressed by plasmids distinct from the amplicon plasmid, this is not necessarily so. A VP16, VHS, and another protein (e.g., a therapeutic protein) or agent (e.g., an siRNA or RNAi) could be expressed from the same plasmid. As shown in Table 1, neither VP16 nor VHS addition to the IE3−/− parental virus influenced GFP titers or amplicon to helper ratios (A:H) compared to pRcCMV-transfected controls.

TABLE 1
VP16 and VHS enhance HSV-1 mutant amplicon packaging

| Helper Virus ^{a} | Plasmid ^{b} | GFU/ml (×10⁶) ^{c} | PFU/ml (×10⁶) ^{d} | A:H Ratio |
|---|---|---|---|---|
| D30EBA | PRcCMV | 2.08 ± 0.07 | 3.23 ± 0.45 | 0.65 ± 0.08 |
| | CMV VHS^{wt} | 1.44 ± 0.32 | 2.73 ± 0.50 | 0.53 ± 0.10 |
| | CMV VP16^{wt} | 2.84 ± 0.60 | 3.17 ± 0.63 | 0.93 ± 0.31 |
| Δ3vhsZ | PRcCMV | 0.13 ± 0.03 | 0.31 ± 0.08 | 0.43 ± 0.07 |
| | CMV VHS^{wt} | 1.02 ± 0.08 ** | 0.58 ± 0.06 | 1.76 ± 0.12 ** |
| 14HΔ3 | PRcCMV | 0.11 ± 0.06 | 0.91 ± 0.22 | 0.12 ± 0.04 |
| | CMV VP16^{wt} | 2.37 ± 0.39 ** | 1.70 ± 0.27 | 1.42 ± 0.30 * |

^{a} HSV-1 mutant viruses used for amplicon packaging as described in the methods section
^{b} Plasmids expressing HSV-1 cDNAs or control vectors (pRcCMV) used to complement amplicon packaging
^{c} Amplicon titers expressed as green fluorescent units determined for unconcentrated virus stocks made using the plasmid HSV4/5lucCMVegfp (* = p < 0.02, ** = p < 0.001)
^{d} Helper titers determined on the RR1 packaging line scored as described in materials and methods.

Also, addition of HMBA, which complements the VP16ⁱⁿ defect, exerted no effect on A:H ratios in these stocks, and, in fact, lowered the total production of virus per well across all treatment groups. Packaging with both 14HΔ3 and Δ3vhsZ mutant helper viruses exhibited marked reduction in titers and A:H ratios, however each was complemented by the co-expression of the respective mutated HSV gene, augmenting amplicon titers between 8- and 21-fold, and improving A:H ratios between 1.5 to 3-fold when compared to D30EBA-packaged groups. These results demonstrate that the restricted replication strategy preferentially facilitates the production of amplicon virus (herpesvirus amplicon particles) over helper virus.

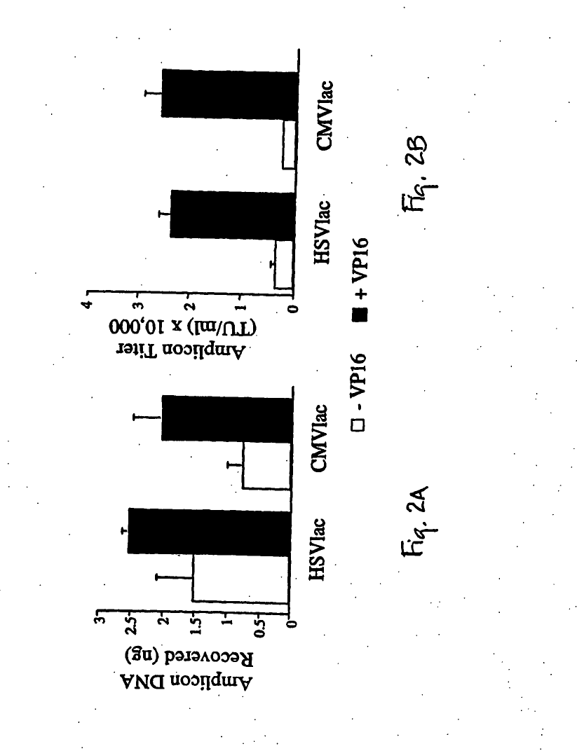

Given the robust increase in amplicon titers produced by VP16 expression via the co-transfected expression plasmid, further analyses were undertaken to understand the mechanism(s) responsible for this phenomenon. To investigate whether VP16 supplementation increased the number of viable infectious amplicon-containing virus particles, real-time quantitative PCR was performed on DNA harvested from Vero monolayers transduced with control or VP16-supplemented packaging stocks. The addition of wild-type VP16 produced a 2-5 fold enrichment in the amount of amplicon DNA-containing viral particles recovered from infected Vero cells for two different amplicon constructs (FIG. 2A). Since HSV variants deleted of VP16 activity make comparable amounts of viral DNA compared to wild-type virus (Weinheimer et al., J. Virol. 66:258-269, 1992), these data suggest that VP16 facilitates the encapsidation of replicated amplicon DNA in the viral nucleocapsid. However, a 5-fold induction does not account for the full augmentation in amplicon titers observed in prior experiments. During the course of viral infection, VP16 is delivered with the herpes genome and stimulates transcription of HSV immediate early genes by binding cognate TAATGARAT (SEQ ID NO:2) elements present in upstream promoter regions. To investigate whether the supplemented VP16 effect on HSVlucCMVegfp titers was mediated through transactivation of cis elements in IE 4/5 promoter present in the dual-reporter vector, we compared plasmid reporter constructs containing (HSVlac) or deleted (CMVminOrislac) of TAATGARAT (SEQ ID NO:2) elements in the VP16 co-transfection paradigm using the helper virus 14ΔA3 (FIG. 2B). In both cases, restoration of VP16 function enriched the number of cells with β-galactosidase activity nearly ten-fold. Therefore, in addition to acting as a transcriptional activator, these data demonstrate that VP16 enhances the packaging of viral genome into infectious particles. VP16 may alter the profile of histone acetylation of VP16-responsive transcriptional complexes, thereby enhancing the transcriptional competence of in vitro templates (Utley et al., Nature 394:498-502, 1998). This prompted a comparison of the reporter activity of amplicon vectors packaged with D30EBA, 14HΔ3 or 14HΔ3vhsZ helper virus in NIH 3T3 monolayers treated with the de-acetylase inhibitor trichostatin A (TSA). TSA enhanced GFP titers, an effect that was most pronounced in stocks packaged with the VP16 linker-insertion mutant helper virus, 14HΔ3vhsZ. Interestingly, this effect was inhibited by the delivery of exogenous wild-type VP16 (Table 2, fourth row).

TABLE 2
TSA enhances amplicon titers in VP16ⁱⁿ packaged stocks

| Helper Virus ^{a} | −TSA ^{b}(GFU/ml) | +TSA ^{c}(GFU/ml) | Fold Δ |
|---|---|---|---|
| D30EBA | 2.30 ± 0.56 × 10⁸ | 3.23 ± 0.60 × 10⁸ | 1.4× |
| 14HΔ3 | 0.30 ± 0.09 × 10⁸ | 0.63 ± 0.19 × 10⁸ | 2.1× |
| 14HΔ3vhsZ | 0.16 ± 0.04 × 10⁷ | 0.60 ± 0.16 × 10⁷ | 3.8× |
| 14HΔ3vhsZ + VP16^{wt} | 3.72 ± 0.67 × 10⁷ | 5.34 ± 0.96 × 10⁷ | 1.4× |

^{a} HSV-1 mutant viruses used for amplicon packaging as described in the methods
^{b, c} Cultures were either treated with or without TSA (0.5 μg/ml) at the time of infection with amplicon vectors as listed in the previous column and were allowed to express for 24 hours prior to GFP amplicon-based titering. Amplicon titers are expressed as green forming units per ml (GFU/ml).

These studies suggest that, in the absence of either wild-type VP16 activity or trichostatin A, histone de-acetylases (present at the time of vector packaging and/or active within the infected target cell) silence the amplicon transcription unit. In aggregate, these data show that VP16 acts independently of canonical cis elements to augment amplicon titers by increasing the production of mature, infectious amplicon-containing virions, and by promoting the transcriptional “competence” of the amplicon DNA template.

### Example 2

### VP16-Supplemented Amplicon Vector Stocks Exhibit Reduced Toxicity on Neuronal Cultures

Attenuation of VP16-driven IE gene expression by linker-insertion mutagenesis significantly improves toxicity profiles for the 14HΔ3 and 14HΔ3vhsZ viruses when tested on cultured cell lines (Johnson et al., J. Virol. 68:6347-6362, 1994). More generally, the toxicity profile of the vectors was improved by dampening IE genes. However, previously published studies have demonstrated that, compared to cultured cell lines, dissociated cortical cultures exhibit additional susceptibility to infection with helper virus-containing stocks (Ho et al., J. Neurosci. Meth. 57:205-215, 1995). Therefore, packaging and evaluatuation of the toxicity profiles of VP16-attenuated amplicon vectors on purified cortical neurons was carried out. 14HΔ3 and Δ3vhsZ helper viruses produced amplicon titers comparable to D30EBA-packaged vectors, whereas the triple mutant (14HΔ3vhsZ) was relatively inefficient (Table 3, row 6).

TABLE 3
VHS complements amplicon packaging using mutant HSV-1 viruses.

| Helper ^{a} Virus | Plasmid ^{b} | GFU/ml (×10⁵) ^{c} | PFU/ml (×10⁵) ^{d} | A:H Ratio |
|---|---|---|---|---|
| Δ3vhsZ | pRcCMV | 0.68 ± 0.44 | 8.17 ± 0.76 | 0.08 ± 0.04 |
| | vhs^{wt} | 1.66 ± 0.28 * | 8.87 ± 0.90 | 0.19 ± 0.05 * |
| | vhsM384 | 2.15 ± 0.60 * | 4.83 ± 0.61 | 0.44 ± 0.07 ** |
| | vhsSC243 | 1.86 ± 0.20 * | 4.77 ± 1.14 | 0.40 ± 0.08 ** |
| | pBSIIvhs^{wt} | 5.44 ± 1.05 * | 6.57 ± 1.64 | 0.84 ± 0.07 *** |
| 14HΔ3vhsZ | pRcCMV | 0.02 ± 0.01 | 0.77 ± 0.15 | 0.02 ± 0.01 |
| | VP16/vhs^{wt} | 3.19 ± 0.24 ** | 2.70 ± 0.60 | 1.21 ± 0.21 ** |
| | VP16/vhsM384 | 5.76 ± 3.55 | 4.60 ± 0.26 | 1.30 ± 0.47 * |
| | VP16/vhsSC243 | 2.70 ± 0.72 * | 3.90 ± 1.82 | 0.82 ± 0.44 |
| | VP16/pBSIIvhs | 4.67 ± 0.41 ** | 5.00 ± 0.69 | 0.94 ± 0.06 *** |

^{a} HSV-1 mutant viruses used for amplicon packaging as described in the methods section
^{b} Control (pRcCMV), VP16, VHS and point-mutant VHS expression vectors used to complement amplicon packaging were delivered by transfection along with the reporter plasmid HSV4/5lucCMVegfp
^{c} Titers expressed as green fluorescent units/ml. * = p < 0.05, ** = p < 0.01, *** = p < 0.001)
^{d} Helper titers are expressed as plaque forming units/ml.

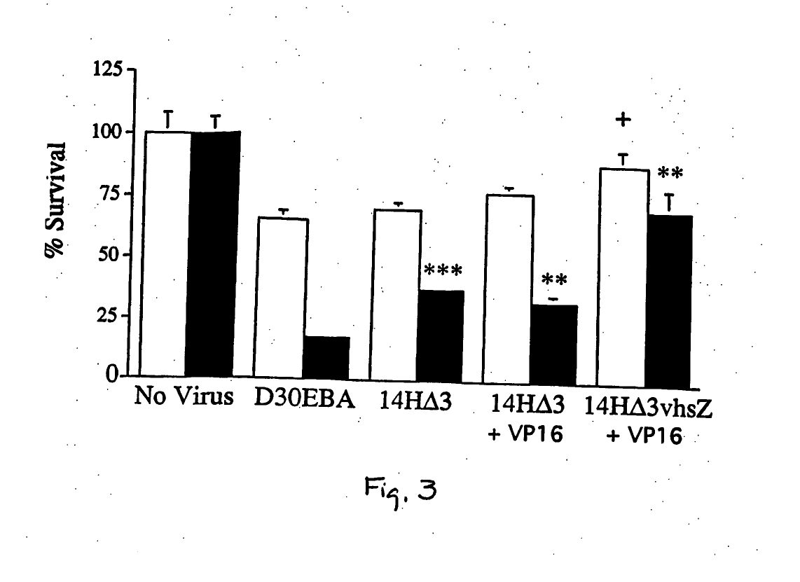

Because the titers for 14H□3vhsZ vectors were log orders below the other vector-virus combinations, evaluating vector toxicity at a multiplicity of infection (MOI) of 2.0 was impractical. DIV 6 neuronal cultures were transduced at an MOI of 2.0 and, survival of replicate cultures was determined at 24 and 72 hours post-infection (FIG. 3). The IE3 mutant D30EBA exhibited reductions in survival at both time points, consistent with the accumulated effect of residual immediate-early gene expression. Interestingly, despite the gains in amplicon titer upon addition of VP16 to 14H□3-packaged vectors, viability was comparable between the two groups. Most notable was the reduction in toxicity imparted by the 14H□13vhsZ-packaged vectors supplemented with wild-type VP16. The marked difference in survival between helper viruses containing wild-type vhs and the 14H□3vhsZ group suggested that VHS contributed to toxicity.

To test whether this loss in viability could be attributed in part to HSV IE gene expression, viral stocks were exposed to 200 Joules/cm² of UV light prior to infection. Titering by plaque assay confirmed a reduction in helper titers between 800-8000 fold in the four treatment groups. In MTS assays, UV inactivation produced equivalent post-transduction survival curves for cortical cultures transduced at an MOI of 2.0 with D30EBA, 14HΔ3, and 14HΔ3vhsZ, regardless of VP16 supplementation. These results support the contention that vector toxicity stems primarily from a virus-derived source (i.e., either helper virus IE gene expression or preformed viral protein(s)) rather than from co-purified excitotoxins released from the packaging cell line at the time of virus production.

### Example 3

### The Role of VHS in Amplicon Titers and Toxicity

VHS participates in the structural integrity of the infectious virion and catalyzes the non-specific degradation of host and viral mRNA species. It is also dispensable for viral replication (Reak and Frenkel, J. Virol. 46:498-512, 1983; Kwong et al., J. Virol. 62:912-921, 1988). To test whether VHS's ability to degrade mRNA is important for enhanced amplicon titers or influences post-infection toxicity, the point mutants vhsM384 and vhsSC243 were compared to wild-type VHS in the restricted complementation-packaging assay (Table 3). Although the point mutants lack mRNAse activity, their inclusion within infectious virus particles is preserved (Jones et al., J. Virol. 69:4863-4871, 1995). When compared to transfected controls, mutant VHS improved amplicon titers as well as, if not better than, wildtype VHS, suggesting that mRNAse activity is dispensable for efficient amplicon packaging.

VHS delivered at the time of transduction disrupts host mRNA stability, while the de novo expression of VHS at the mid-point of the viral life-cycle directs tegument assembly limiting VP16-mediated transcriptional activity thereby redirecting viral gene expression towards early and late viral gene products (Schmelter et al., J. Virol. 70:2124-2131, 1996). In light of these considerations, we investigated whether control over the timing as well as overall levels of VHS expression would influence the efficiency of the amplicon packaging process. Since the CMV promoter supports high levels of transgene expression, a VHS expression plasmid containing the native HSV-1 transcriptional control elements was created. As shown in Table 3, inclusion of the flanking sequences boosted amplicon titers as effectively as CMV-VHS packaged control, irrespective of the helper virus used during packaging. Thus, driving a VHS gene with its own promoter may support packaging of amplicons better than a more highly engineered construct (such as one in which a CMV regulatory element drives VHS). Having identified that the restricted replication protocol could augment viral titers for double deletion helper viruses, it was hypothesized that the defect in virus production exhibited by the triple deletion 14HΔ3vhsZ could be overcome using a similar strategy. Addition of VHS and VP16 separately enhanced the total amplicon production and improved A:H ratios. Importantly, co-transfection of VP16 and VHS to the 14HΔ3v/is Z background produced an additive effect (Table 3, row 6 vs. 7).

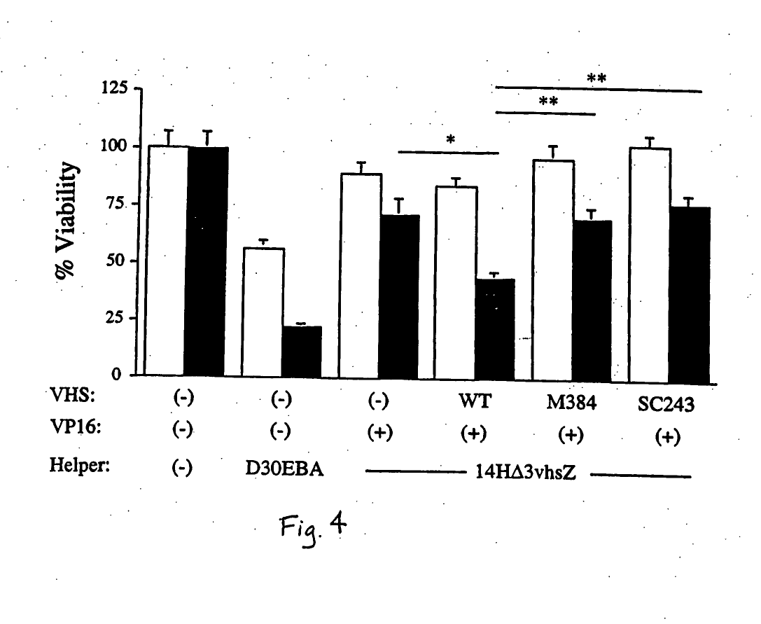

To address the neurotoxicity imparted by the virion host shutoff protein, amplicon stocks packaged with the 14HΔ3vhsZ helper on cultured cell lines were tested. The behavior of the Δ3vhsZ-packaged vectors was not investigated as the virus elicits significantly greater cytopathic effect on the host cell due to unopposed VP16-mediated immediate early genes expression (Johnson et al., J. Virol. 68:6347-6362, 1994). When tested on primary cortical neuronal cultures, vectors packaged using 14HΔ4vhsZ were superior to control D30EBA-generated amplicon stocks at both 24- and 72-hours post infection. Substitution of wild-type VHS with M384 and SC243 point mutants conferred additional benefit, and survival approximated that of uninfected control cultures at the earlier time point (FIG. 4).

### Example 4

### Development of Integrating HSV-1 Amplicon Vectors via Adaptation of the Sleeping Beauty Transposition System

In the studies that follow, the Tc1-like Sleeping Beauty (SB) transposon system was combined with the amplicon to engineer a novel integrating vector. Two vectors were constructed: one containing an RSV promoter-driven β-galactosidase-neomycin (βgeo) fusion flanked by the SB terminal repeats (HSVT-βgeo), and a second containing the SB transposase gene transcriptionally controlled by the HSV immediate-early 4/5 gene promoter (HSVsb). Co-transduction of BHK cells, murine primary cultures, adult striata, and neonatal brain resulted in integration of the transposable transgene (transgenon) and extension of expression duration in vivo. This new HSV amplicon iteration will protract expression profiles for gene-based amelioration of disease. The methods used to conduct these studies and the results are described in more detail below.

The Sleeping Beauty transposon system. Sleeping Beauty is a synthetic transposon system that was constructed from defective units of a Tc1-like fish element. It consists of a 1.6-kb element flanked by 250-bp inverted repeats and encodes for a single protein, the Sleeping Beauty transposase. The reconstructed enzyme catalyzes transposition of ITR-flanked genetic units from one genomic locus to another. In addition, Sleeping Beauty can facilitate integration of naked DNA from episomes into human and mouse chromosomes (Ivics et al., Cell 91:501-510, 1997; Luo et al., Proc. Natl. Acad. Sci. USA 95:10769-10773, 1998; Yant et al., Nat. Genet. 25:35-41, 2000).

HSV amplicon particles. As noted above, the HSV amplicon is a versatile vector for gene delivery to post-mitotic cells. Because it is inherently neurotropic and easy to manipulate, the amplicon can be used to administer therapeutic agents to neurons within (or from) the central and peripheral nervous systems. Amplicons efficiently transduce mitotically active cells to achieve transient expression of proteins in vitro and in vivo. Amplicon particles made by the methods described here are particularly advantageous because they are stably maintained within cells, where they mediate long-term gene expression. Thus, expression can remain robust in dividing cell types of the CNS, such as stem-like cells or cells of the glial lineage; integration-competent viral vectors that insert into transcriptionally active chromosomal regions exhibit prolonged transgene expression profiles.

Cell culture. Baby hamster kidney (BHK) cells were maintained as described in Lu et al. (Hum. Gene Ther. 6:421430, 1995). The NIH-3T3 mouse fibroblast cell line was originally obtained from American Type Culture Collection and maintained in Dulbecco's modified Eagle medium (DMEM) supplemented with 10% fetal bovine serum (FBS), 100 units/ml penicillin, and 100 μg/ml streptomycin. Primary cortical neurons were harvested from E15 mice and were prepared as described by Brewer et al. (J. Neurosci. Res. 42:674-683, 1995). Cortices were dissociated initially by trypsinization (0.25% trypsin/EDTA) for 15 min at 37° C. and washed twice with HBSS containing Ca²⁺ and Mg²⁺. Cells were mechanically dissociated further using a serologic pipette and resuspended in serum-free Neurobasal® plating medium containing 0.5 mM L-glutamine, 3.7 μg/ml L-glutamate and 2% B-27 supplement (Life Technologies, Gaithersburg, Md.). Cultures were maintained at 37° C. in a 6% CO₂ environment.

Amplicon construction. The Sleeping Beauty transposase encoding sequence was removed from the pCMV-SB plasmid (Yant et al. Nat. Genet. 25:35-41, 2000); kindly provided by Dr. M. Kay) by XhoI-SalI digestion and cloned into the SalI site of pHSVPrPUC (Geller and Freese, Proc. Natl. Acad. Sci. USA 87:1149-1153, 1990) to create pHSVsb. The integration-competent transcription cassette from pT-βgeo (Yant et al. Nat. Genet. 25:35-41, 2000) was removed using KpnI and VspI, blunted, and cloned into the blunted Hind III site of pHSVminOriSmc amplicon to create pHSVT-βgeo. In a subset of experiments, the pHSVPrPUC amplicon was employed as an empty vector control.

Helper virus-free HSV amplicon packaging. Amplicon vectors were packaged as described herein (see also Bowers et al., Gene Ther. 8:111-120, 2001). Viral pellets were resuspended in 100 μl PBS and stored at −80° C. until use. Vectors were titered as described previously (Bowers et al., Mol. Ther. 1:294-299, 2000).

Real-time Quantitative PCR Analyses. To isolate total DNA for quantitation of amplicon genomes in transduced cells or brain tissue, isolates were lysed in 100 mM potassium phosphate (pH 7.8) and 0.2% Triton X-100. An equal volume of 2× Digestion Buffer (0.2 M NaCl, 20 mM Tris-Cl (pH 8), 50 mM EDTA, 0.5% SDS, 0.2 mg/ml proteinase K) was added to the lysate and the sample was incubated at 56° C. for four hours. Samples were processed further by one phenol:chloroform, one chloroform extraction, and a final ethanol precipitation. Total DNA was quantitated and 25 ng of total DNA was analyzed in a PE7700 quantitative PCR reaction using a designed lacZ-, or Sleeping Beauty transposase gene-specific primer/probe combination multiplexed with an 18S rRNA-specific primer/probe set.

The lacZ probe sequence was 5′-6FAM-ACCCCGTACGTCTTCCCGAGCG-TAMRA-3′ (SEQ ID NO:10); the lacZ sense primer sequence was 5′GGGATCTGCCATTGTCAGACAT-3′ (SEQ ID NO:4); and the lacZ antisense primer sequence was 5′-TGGTGTGGGCCATAATTCAA-3′ (SEQ ID NO:5). The Sleeping Beauty probe sequence was 5′-6FAM-AAGAAGCCACTGCTCCAAAACCGACA-TAMRA-3′ (SEQ ID NO:11); the Sleeping Beauty sense primer sequence was 5′-CCACAGTAAAACGAGTCCTATATCGA-3′ (SEQ ID NO:12); and the Sleeping Beauty antisense primer sequence was 5′-TGCAAACCGTAGTCTGGCTTT-3′ (SEQ ID NO:13). The 18S rRNA probe sequence was 5′-MAX-TGCTGGCACCAGACTTGCCCTC-TAMRA-3′ (SEQ ID NO:14); the 18S sense primer sequence was 5′-CGGCTACCACATCCAAGGAA-3′ (SEQ ID NO:15); and the 18S antisense primer sequence was 5′-GCTGGAATTACCGCGGCT-3′ (SEQ ID NO:16).

Analysis of integrated vector sequences. Inverse PCR was utilized for analysis of junction fragments as previously described above by Luo et al., using the identical three sets of nested primers that were designed for both the left (IR/DR-L) and right ends of the ITR (IR/DR-R) (Proc. Natl. Acad. Sci. USA 95:10769-10773, 1998). Briefly, genomic DNA was purified from amplicon-transduced primary neuronal cultures at Day 9 post-transduction, digested with Sau3AI, and ligated with T4 DNA ligase. Samples were subsequently subjected to three rounds of PCR using the nested primer sets. Amplified products arising from the third PCR reaction were ligated into the pGEMT-Easy cloning vector and sequenced using the dye terminator method.

Stereotactic delivery of amplicon vectors into adult mice. Eight to ten week-old male C57BL/6 mice (Jackson Laboratories) were anesthetized with Avertin (300 mg/kg) during stereotactic intrastriatal injections. After positioning in a mouse stereotactic apparatus (ASI Instruments, Warren, Mich.) the skull was exposed via a midline incision, and burr holes were drilled over the designated coordinates (Bregma, 0 mm; lateral, 2.0 mm; ventral, 3.0 mm). A 33-gauge needle was gradually advanced to the desired depth over a period of five minutes. All injections were performed with a microprocessor controlled pump (UltraMicro-Pump; WPI Instruments, Sarasota, Fla.; Brooks et al., J. Neurosci. Meth. 80:137-147, 1998). HSVsb, HSVPrPUC, and/or HSVT-βgeo (3-6×10⁶ transduction units/ml) in 2.0 μl were injected at a constant rate over a period of five minutes (200 nl/min). Upon completion of injection, the needle was removed over a period of five minutes. Mice were sacrificed 7, 21 and 90 days post-injection for biochemical and immunocytochemical analyses.

Delivery of amplicon vectors into neonatal mice. C3H mice (P1) were anesthetized by inducing a light hyperthermia followed by manual injection of helper-free HSV amplicon virus into the right hemisphere of the brain. Specifically, a 33-gauge needle was carefully positioned above the right hemisphere and slowly advanced the desired depth. HSVsb+HSVT-βgeo or HSVT-βgeo+HSVPrPuc in a total volume of 1 μl was manually injected. The needle was slowly removed, mice were warmed under a heat lamp and returned to their respective dams. Mice were sacrificed 90 days post-injection for immunocytochemical analyses.

Tissue preparation and immunocytochemistry. Injected adult mice were anesthetized at 7, 21, and 90 days post-injection, a catheter was placed into the left ventricle, and intracardiac perfusion was initiated with 10 ml of heparinized saline (5,000 U/L saline) followed by 60 ml of chilled 4% PFA in saline. Brains were extracted and postfixed for one to two hours in 4% PFA at 4° C. Subsequently, brains were cryoprotected in a series of sucrose solutions with a final solution consisting of a 30% sucrose concentration (w/v) in PBS. Twenty-five micron serial sections were cut on a sliding microtome (Micron/Zeiss, Thornwood, N.Y.) and stored in a cryoprotective solution (30% sucrose (w/v), 30% ethylene glycol in 0.1 M phosphate buffer (pH 7.2)) at −20° C. until processed for immunocytochemistry.

Upon removal of cryoprotectant, sections were placed into Costar net wells (VWR, Springfield, N.J.) and incubated for two hours in 0.1 M Tris buffered saline (TBS) (pH 7.6). Two additional 10 minute washes in 0.1 M TBS with 0.25% Triton X-100 (Sigma Chemical Co., St. Louis, Mo.) were performed. Sections were permeabilized in 0.1 M phosphate buffer and 0.4% Triton-X-100 for 5 minutes at 25° C. Non-specific binding sites were blocked using 0.1 M phosphate buffer, 10% normal goat serum and 0.4% Triton-X-100 for one hour at 25° C. Double immunolabeling was performed using anti-β-galactosidase, rabbit IgG Fraction A-11132 (1:2000, Molecular Probes, Eugene, Oreg.), with either mouse anti-Neuronal Nuclei (NeuN) monoclonal antibody (1:200, Chemicon International, Temecula, Calif.), or an anti-Glial Fibrillary Acidic Protein (GFAP)-cy3 conjugate monoclonal antibody clone G-A-5 (1:2000, Sigma, St. Louis, Mo.). Sections were incubated for 48 hours at 4° C. with primary antibodies diluted in 0.1 M phosphate buffer, 1% normal goat serum and 0.4% Triton-X-100. After rinsing in 0.1 M phosphate buffer (5×5 minutes), fluorescent secondary antibodies (fluorescein anti-rabbit IgG (H+ L; 1:200, Vector Laboratories, Burlingame, Calif.), and Rhodamine Red™-X-conjugated* AffiniPure goat anti-mouse IgG (H+ L) (1:200, Jackson Immuno Research Labratories Inc., West Grove, Pa.) diluted in 0.1 M phosphate buffer plus 1% normal goat serum and 0.4% Triton-X-100 were added to the sections and incubated for two hours at 25° C. The sections were rinsed in 0.1 M phosphate buffer, mounted on glass slides with Mowiol, and visualized using a confocal laser scanning microscope (FV 300, Olympus, Melville, N.Y.). All images obtained from immunocytochemical analyses were digitally acquired with a 3-chip color CCD camera at 200× magnification (DXC-9000, Sony, Montvale, N.J.).

Results. The ability of an HSV amplicon vector to deliver a transposable transcription unit for preferential expression in cells of glial origin was examined using a two-vector approach. One amplicon was constructed to express high levels of the Sleeping Beauty transposase (HSVsb) under transcriptional control of the HSV immediate-early 4/5 promoter. The second amplicon served as the substrate vector for the transposase and carried a terminal inverted repeat-flanked transgene segment (termed ‘transgenon’) which expressed a β-galactosidase-neomycin resistance gene fusion under Rous sarcoma virus (RSV) long terminal repeat transcriptional control (HSVT-βgeo). This promoter is widely expressed, but when employed in the context of the CNS imparts expression selectivity to specific regions of the brain Smith et al. J. Virol. 74:11254-11261, 2000). A two-vector strategy was employed since inclusion of both components in one vector would likely lead to transposition events occurring within the packaging cell resulting in inefficient virion generation. The two vectors were packaged separately using a modified helper virus-free method (Bowers et al., Gene Ther. 8:111-120, 2001).

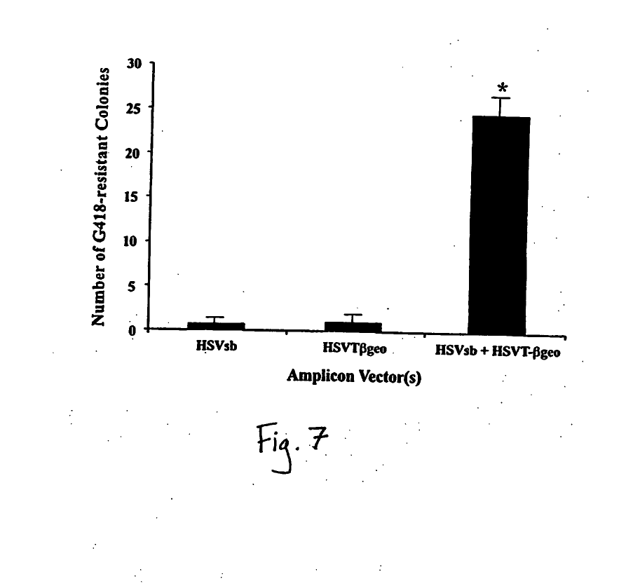

To determine if co-transduction with two amplicon vectors would result in enhanced integration in mitotically active cells, testing was initiated in baby hamster kidney (BHK) cells. BHK cultures were transduced with equivalent virion numbers of HSVsb+HSVPrPUC (empty vector control), HSVT-βgeo+HSVPrPUC, or HSVT-βgeo+HSVsb. Cultures subsequently were placed under G418 selection, and resistant colonies that arose following two weeks of drug selection were stained by X-gal histochemistry and enumerated. Co-transduction of HSVsb or HSVT-βgeo with the empty vector control amplicon resulted in very few numbers of G418-resistant, LacZ⁺ colonies (FIG. 7).

However, co-transduction of HSVsb with HSVT-βgeo greatly increased the numbers of colonies (˜25-fold), indicating that an HSV amplicon-harbored transgenon could be stably maintained and expressed only when briefly exposed to the transposase expressed from HSVsb. The expression kinetics of HSVsb was not measured directly, but based upon previous work with other transgenes expressed from the HSVPrPUC backbone, expression levels are highest at 24-48 hours post-transduction and wane over the succeeding 10 days Jin et al. Hum. Gene Ther: 7:2015-2024, 1996).

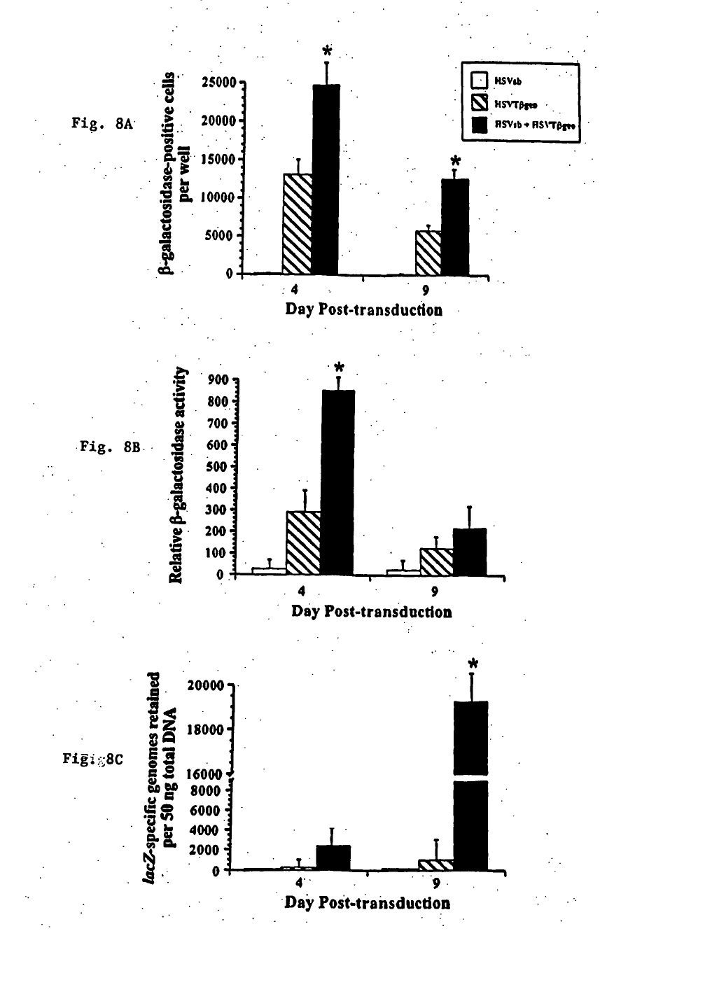

The observations made in actively dividing BHK cells led us to test the new bipartite amplicon platform in primary murine cortical cultures to determine if transposition of the amplicon-bearing transgene unit could occur in cells within the central nervous system. Demonstration of such an event and examination of resultant expression duration profiles and cellular specificity would lead to the design of novel HSV amplicons for treatment of neurodegenerative diseases. Primary cultures were established using B27 medium in the absence of mitotic inhibitors, which has been shown to provide cultures consisting of mainly neuronal cell types with minimal glial contamination. As time in culture increases the population of glial cells is gradually amplified. Primary cultures were established from cortices of embryonic day 15 (E15) C57BL/6 embryos and incubated with equivalent transducing virion numbers of HSVsb, HSVT-βgeo, or both vectors on in vitro day 5 (DIV 5). Treated cultures were processed for X-gal histochemistry, β-galactosidase enzyme activity, and real-time quantitative PCR analysis of the transgenon DNA segment on Days 4 and 9 post-transduction. Enumeration of X-gal-positive cells in each of the treatment groups indicated that cultures receiving both test amplicons exhibited enhanced numbers of transgene-expressing cells on Days 4 and 9 (FIG. 8A). Separate immunocytochemical analysis of cultures indicated that both neurons and glia expressed the βgeo transgene. Analysis of transgene-encoded β-galactosidase enzyme activity by Galacto-Lite™ assay exhibited similar profiles of expression between the three treatment groups on Day 4 but differences in β-galactosidase activity did not reach statistical significance at Day 9 among the groups (FIG. 8B). Interestingly, when total DNA was harvested from transduced cultures using a method favoring the purification of larger molecular weight DNA, the cultures receiving both test amplicons exhibited an increased number of lacZ sequence targets over time as detected by real-time quantitative PCR (FIG. 8C). These results in aggregate suggested that the transgenon segment of the HSVT-βgeo amplicon had mobilized into the host cell genome in an HSVsb-dependent manner that resulted in appreciably enhanced gene expression as compared to HSVT-βgeo alone.

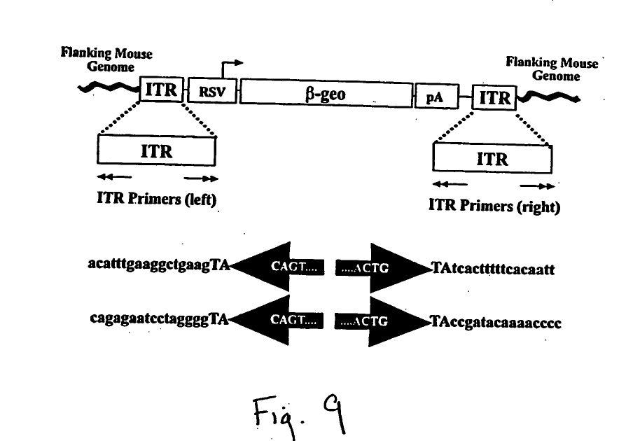

To definitively assess the occurrence of Sleeping Beauty-mediated integration in mouse primary culture cells, we employed inverse PCR as previously described by Luo et al. (Proc. Natl. Acad. Sci. USA 95:10769-10773, 1998) was employed. On Day 9 post-transduction, high molecular weight DNA isolated from primary cultures that had been treated with both HSVsb and HSVT-βgeo was subjected to three rounds of nested PCR. Resultant integration junction PCR products were sequenced and analyzed for identity of novel flanking nucleotide sequences. It was possible to identify several different flanking sequences that corresponded to murine genomic sequence as assessed from BLAST searches (FIG. 9). There did not appear to be a preference for particular integration sites within the genome as determined by the analysis of numerous inverse PCR products.

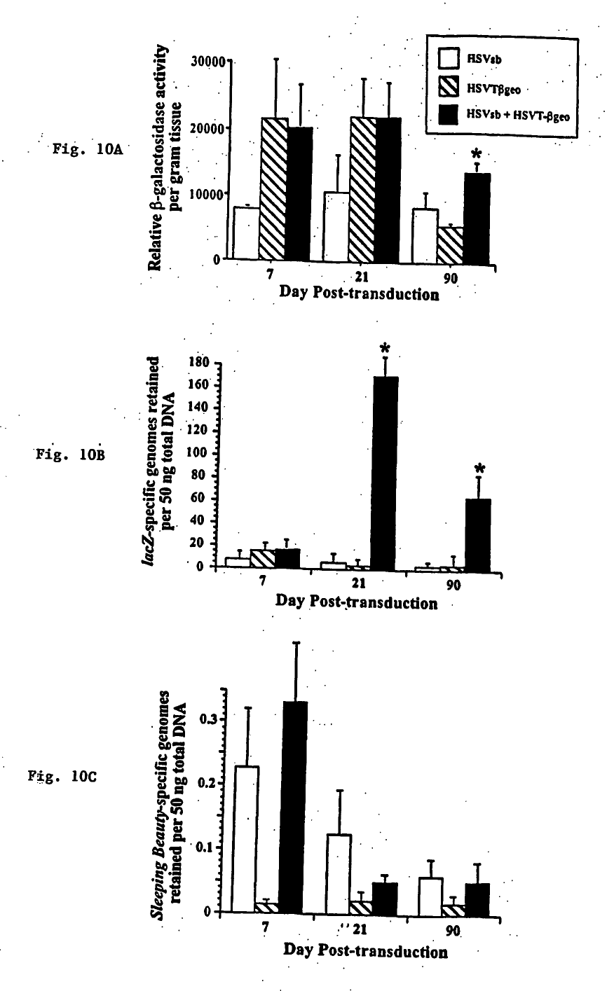

Subsequently the new integration-competent amplicon vector platform was characterized in vivo in the setting of the murine CNS. Three month-old male C57BL/6 mice were transduced with equivalent virion numbers of HSVsb, HSVT-βgeo, or both vectors and were processed for β-galactosidase enzyme activity, real-time quantitative PCR analyses, and immunocytochemistry on Days 7, 21, and 90 post-transduction. The empty vector, HSVPrPUC, was used in the single vector treatments for equilibration of virus particle input. The temporal expression pattern of β-galactosidase was indistinguishable for animals receiving HSVT-βgeo alone and those receiving HSVsb plus HSVT-βgeo on Days 7 and 21 post-transduction (FIG. 10A). At Day 90, there existed a statistically significant difference in transgene expression levels between these two groups as well as the HSVsb-transduced mice. When transgene DNA retention analyses were performed on high molecular weight nucleic acid purified from the injection site, greatly enhanced numbers of transgenon-specific sequences were detected only in animals receiving both HSVsb and HSVT-βgeo amplicons (FIG. 10B). To confirm that only the T-βgeo transgenon segment co-segregated with genomic DNA, quantitative real-time PCR was performed for Sleeping Beauty transposase gene sequences that are harbored in the HSVsb amplicon. The transposase-specific sequences were readily detectable on Day 7 but were difficult to detect above background signals on Days 21 and 90 post-transduction, indicating that transposition events were specific to the transgenon-carrying amplicon vector, HSVT-βgeo (FIG. 10C). As stated above, in vivo amplicon administration was performed using equal virion numbers of HSVsb and HSVT-βgeo (or the control HSVPrPUC amplicon, where appropriate).

The in vivo biochemical data suggested that cells of the murine CNS were amenable to transposition of a mobilization-competent transcription unit from an amplicon into the cellular genome. To identify the cell type(s) harboring and expressing the transgenon fluorescent immunocytochemistry was utilized to visualize lacZ in conjunction with the neuronal marker, NeuN, or the glial cell marker, GFAP. Transgenon-derived β-galactosidase expression consistently localized to GFAP-positive cells in mice receiving HSVT-βgeo or the HSVsb/HSVT-βgeo combination, and was rarely, if ever, detected in NeuN-positive neurons (n=12). Differences in transgene expression duration existed amongst the various treatment groups. It was possible to detect lacZ expression only in brains receiving the combined HSVsb/HSVT-βgeo amplicon treatment at Day 90 post-transduction, further confirming the results obtained from enzyme activity assays (FIG. 10A). Performance of titration studies by varying either amplicon component did not alter cell type specificity of transgenon expression.

To examine the potential applicability of the new integrating system in perinatal gene transfer paradigms for therapeutic applications or the creation of novel degenerative disease models, the new amplicon vector platform was administered to the CNS of newborn mice. One day-old (P0) C3H mice were transduced with HSVT-βgeo alone, or both HSVsb and HSVT-βgeo, and were processed for fluorescent immunocytochemistry on Day 90 post-transduction. As with the adult C57BL/6 animals, striata from C3H mice transduced at P0 exhibited lacZ transgene expression only in GFAP-positive cells. Transgenon expression at Day 90 was dependent upon co-transduction of the HSVsb and HSVT-βgeo amplicons, as animals receiving only HSVT-βgeo did not exhibit any detectable lacZ expression at this time point. In aggregate, these results indicate that this new integrating HSV amplicon vector system extends the utility of this gene delivery platform to provide prolonged transgene expression within cells of the CNS that were once refractory to stable amplicon-mediated expression.

1. A method of delivering a therapeutic agent to a patient, the method comprising administering to the patient a therapeutically effective amount of a herpesvirus amplicon particle generated by a cell that stably expresses a herpes simplex virus (HSV) immediate early 3 (IE3) gene and that is:
(a) infected with a helper virus comprising (i) a mutation in a sequence encoding a VP16 or virion host shutoff (VHS) protein, wherein the mutation reduces the activity of the encoded VP16 or VHS protein; (ii) all of the required HSV structural proteins; and (iii) a herpesvirus cleavage/packaging site;
(b) transfected with a first plasmid comprising (i) a sequence that when transcribed and, optionally, translated, encodes the therapeutic agent and (ii) a herpesvirus origin of replication (ori); and
(c) transfected with a second plasmid comprising a sequence that encodes VP16 (in the event the helper virus comprises a mutation in a sequence encoding VP16) or VHS (in the event the helper virus comprises a sequence encoding VHS).

2. The method of claim 1, further comprising (d): transfected with a third plasmid that encodes a transposase or a biologically active fragment or other mutant thereof.

3. The method of claim 1, wherein the cell is autologous to the patient.

4. The method of claim 3, wherein the patient has been diagnosed with cancer, and the cell is a cancer cell.

5. A method of delivering a therapeutic agent to a patient, the method comprising administering to the patient a therapeutically effective number of cells that stably express an HSV IE3 gene and that comprise:
(a) a helper virus comprising (i) a mutation in a sequence encoding a VP16 or virion host shutoff (VHS) protein, wherein the mutation reduces the activity of the encoded VP16 or VHS protein; (ii) all of the required HSV structural proteins; and (iii) a herpesvirus cleavage/packaging site;
(b) a first plasmid comprising (i) a sequence that when transcribed and, optionally, translated, encodes the therapeutic agent and (ii) a herpesvirus origin of replication (ori); and
(c) a second plasmid comprising a sequence that encodes VP16 (in the event the helper virus comprises a mutation in a sequence encoding VP16) or VHS (in the event the helper virus comprises a sequence encoding VHS); and/or
(d) a herpesvirus amplicon particle generated from the components listed in (a)-(c).

6. The method of claim 5, wherein the cell is autologous to the patient.

7. The method of claim 5, wherein the patient has been diagnosed as having a leukemia and the therapeutic agent upregulates the expression of a co-stimulatory molecule.

8. A method of generating a herpesvirus amplicon particle, the method comprising
(a) providing a cell permissive for herpesvirus propagation;
(b) infecting the cell with a helper virus comprising a mutation that diminishes the activity of a VP16 or VHS protein;
(c) transfecting the cell with a first plasmid comprising an HSV origin of replication, an HSV cleavage/packaging signal, and a heterologous transgene; and
(d) transfecting the cell with a second plasmid comprising a sequence that encodes a protein that is, or is functionally equivalent to, VP16 (in the event the helper virus comprises a mutation that diminishes the activity of VP16) or VHS (in the event the helper virus comprises a mutation that diminishes the activity of VHS).

9. The method of claim 8, further comprising step (e): transfecting the cell with a vector comprising a sequence encoding an enzyme that facilitates insertion of the transgene into the genome of the cell.

10. A herpesvirus amplicon particle generated by the method of claim 8.

11. A cell or a cell of a cell line comprising the herpesvirus amplicon particle of claim 10.

12. A plasmid comprising a sequence that encodes a VP16 or VHS protein.

13. A kit comprising a herpesvirus amplicon particle generated by the method of claim 8 and instructions for use.

14. The method of claim 1, wherein the herpesvirus is an alpha herpesvirus or an Epstein-Barr virus.

15. The method of claim 14, wherein the alpha herpesvirus is a Varicella-Zoster virus, a pseudorabies virus, or a herpes simplex virus.

16. The method of claim 1, wherein the mutation is a mutation in VHS that inhibits the interaction between VP16 and VHS.

17. The method of claim 1, wherein the mutation is a mutation in VHS that inhibits the ability of VHS to degrade mRNA.

18. The method of claim 1, wherein the mutation is a mutation in the VHS sequence comprising residues 237-489.

19. The method of claim 1, wherein the VHS protein is an HSV-1 virion host shutoff protein, an HSV-2 virion host shutoff protein, an HSV-3 virion host shutoff protein, bovine herpesvirus 1 virion host shutoff protein, bovine herpesvirus 1.1 virion host shutoff protein, gallid herpesvirus 1 virion host shutoff protein, gallid herpesvirus 2 virion host shutoff protein, suid herpesvirus 1 virion host shutoff protein, baboon herpesvirus 2 virion host shutoff protein, pseudorabies virus virion host shutoff protein, cercopithecine herpesvirus 7 virion host shutoff protein, meleagrid herpesvirus 1 virion host shutoff protein, equine herpesvirus 1 virion host shutoff protein, or equine herpesvirus 4 virion host shutoff protein.

20. The method of claim 1, wherein the VHS protein is operatively coupled to its native transcriptional control elements.

21. The method of claim 1, wherein the VP16 protein is HSV1 VP16, HSV-2 VP16, bovine herpesvirus 1 VP16, bovine herpesvirus 1.1 VP16, gallid herpesvirus 1 VP16, gallid herpesvirus 2 VP16, meleagrid herpesvirus 1 VP16, or equine herpesvirus 4 VP16.

22. The method of claim 1, wherein the therapeutic agent is a protein or an RNA molecule.

23. The method of claim 1, wherein the therapeutic agent is an antisense RNA molecule, an siRNA, or a ribozyme.

24. The method of claim 1, wherein the protein is a receptor, a signaling molecule, a transcription factor, a growth factor, an apoptosis inhibitor, an apoptosis promoter, a DNA replication factor, an enzyme, a structural protein, a neural protein, a heat shock protein, or a histone.

25. The method of claim 1, wherein the protein is an immunomodulatory protein, a tumor-specific antigen, or an antigen of an infectious agent.

26. The method of claim 1, wherein the immunomodulatory protein is a cytokine or a costimulatory molecule.

27. The method of claim 26, wherein the cytokine is an interleukin, an interferon, or a chemokine.

28. The method of claim 26, wherein the costimulatory molecule is a B7 molecule or CD40L.

29. The method of claim 25, wherein the tumor-specific antigen is a prostate specific antigen.

30. The method of claim 25, wherein the infectious agent is a virus or a prion protein.

31. The method of claim 30, wherein the virus is a human immunodeficiency virus.

32. The method of claim 25, wherein the antigen of an infectious agent is gp120.

33. The method of claim 25, wherein the antigen of an infectious agent is a bacterial or parasitic antigen.

34. The method of claim 1, wherein the first plasmid further comprises a promoter.

35. The method of claim 1, wherein the cell is a neuron, a blood cell, a hepatocyte, a keratinocyte, a melanocyte, a neuron, a glial cell, an endocrine cell, an epithelial cell, a muscle cell, a prostate cell, or a testicular cell or a germ cell.

36. The method of claim 1, wherein the cell is a malignant cell.

37. The method of claim 1, wherein the patient has Creutzfeld-Jacob Disease.

38. The method of claim 1, wherein the patient has, or is at risk for developing, hearing loss, and the transgene encodes a protein that exerts a protective effect on spiral ganglion neurons.

39. The method of claim 38, wherein the transgene encodes a neurotrophin.

40. The method of claim 39, wherein the neurotrophin is neurotrophin-3.

41-55. (canceled)


SEQUENCE LISTING (15 sequences)

SEQ ID NO: 1 - DNA, 18 bp, Artificial Sequence
  Primer
gctggaattaccgcggct

SEQ ID NO: 2 - DNA, 9 bp, Artificial Sequence
  VP16 binding element
taatgarat

SEQ ID NO: 3 - DNA, 8 bp, Artificial Sequence
  VP16 binding element
tatgarat

SEQ ID NO: 4 - DNA, 22 bp, Artificial Sequence
  Primer
gggatctgccattgtcagacat

SEQ ID NO: 5 - DNA, 20 bp, Artificial Sequence
  Primer
tggtgtgggccataattcaa

SEQ ID NO: 6 - DNA, 22 bp, Artificial Sequence
  Probe sequence
accccgtacgtcttcccgagcg

SEQ ID NO: 7 - DNA, 21 bp, Artificial Sequence
  Primer
gcttctttgcagctccttcgt

SEQ ID NO: 8 - DNA, 17 bp, Artificial Sequence
  Primer
ccagcgcagcgatatcg

SEQ ID NO: 9 - DNA, 20 bp, Artificial Sequence
  Probe sequence
cgccaccagttcgccatgga

SEQ ID NO: 10 - DNA, 22 bp, Artificial Sequence
  Probe sequence
accccgtacgtcttcccgagcg

SEQ ID NO: 11 - DNA, 26 bp, Artificial Sequence
  Probe sequence
aagaagccactgctccaaaaccgaca

SEQ ID NO: 12 - DNA, 26 bp, Artificial Sequence
  Primer
ccacagtaaaacgagtcctatatcga

SEQ ID NO: 13 - DNA, 21 bp, Artificial Sequence
  Primer
tgcaaaccgtagtctggcttt

SEQ ID NO: 14 - DNA, 22 bp, Artificial Sequence
  Probe sequence
tgctggcaccagacttgccctc

SEQ ID NO: 15 - DNA, 20 bp, Artificial Sequence
  Primer
cggctaccacatccaaggaa